(19) United States
(12) Patent Application Publication
(10) Pub. No.: US 2010/0190814 A1
(43) Pub. Date: Jul. 29, 2010

(54) SPIROINDOLINONE DERIVATIVE PRODRUGS

(76) Inventors: Li Chen, Shanghai (CN); Xingchun Han, Shanghai (CN); Lisha Wang, Shanghai (CN); Min Wang, Shanghai (CN); Song Yang, Shanghai (CN); Zhuming Zhang, Hillsborough, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/685,750

(22) Filed: Jan. 12, 2010

Related U.S. Application Data
(60) Provisional application No. 61/147,176, filed on Jan. 26, 2009.

(51) Int. Cl. A61K 31/438; C07D 471/10

(52) U.S. Cl. 514/278; 546/17

(57) ABSTRACT

There are provided compounds of the formula

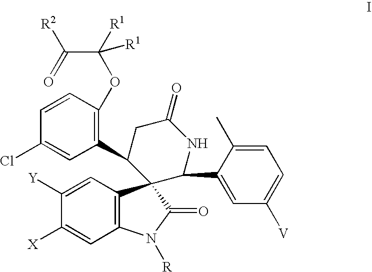

and pharmaceutically acceptable salts and esters and enantiomers thereof
wherein X, Y, V, R¹, R² and R are as described herein.

The compounds have utility as antiproliferative agents, especially, as anticancer agents.

### PRIORITY TO RELATED APPLICATION(S)

This application claims the benefit of U.S. Provisional Application No. 61/147,176, filed Jan. 26, 2009, which is hereby incorporated by reference in its entirety.

### SUMMARY OF THE INVENTION

The present invention relates to spiroindolinone derivatives of the formula

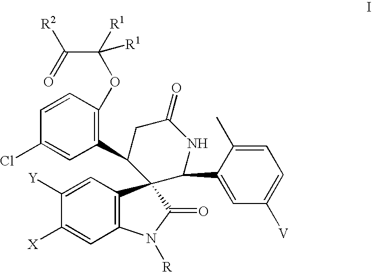

and pharmaceutically acceptable salts and esters and enantiomer thereof
wherein X, Y, V, R¹, R² and R are as described herein.

The compounds have utility as prodrugs leading to other anticancer agents.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

A series of spiroindolinone as antagonists of MDM2 has previously been disclosed in J. Am. Chem. Soc., 2005, 127, 10130 and also in US-2007-0213341-A1 published Sep. 13, 2007.

A prodrug is in most cases a pharmacologically inactive derivative of a parent drug molecule that requires spontaneous or enzymatic transformation within the body in order to release the active drug, and that has improved delivery properties over the parent drug molecule. It has been shown that a molecule with optimal structural configuration and physicochemical properties for eliciting the desired therapeutic response at its target site does not necessarily possess the best molecular form and properties for its delivery to its point of ultimate action. Usually, only a minor fraction of doses administered reach the target area and since most agents interact with non-target sites as well, an inefficient delivery may result in undesirable side effects. This fact of differences in transport and in situ effect characteristics for many drug molecules is the basic reason why bioreversible chemical derivatization of drugs, i.e., prodrug formation is a means by which a substantial improvement in the overall efficacy of drugs can often be achieved. Prodrugs are designed to overcome pharmaceutically and/or pharmacokinetically based problems associated with the parent drug molecule that would otherwise limit the clinical usefulness of the drug.

The advantage of a prodrug lies in its physical properties, such as enhanced water solubility for parenteral administration or oral administration compared to the parent drug, or it enhances absorption from the digestive tract, or it may enhance drug stability for long-term storage. Compounds of formula IA have limited oral bioavailability. It was therefore useful to find derivatives of the compounds of formula IA to render these compounds suitable for oral administration. The present invention provides spiroindolinone derivative prodrugs whose in vivo degradation/cleavage products (formula IA) are small molecule inhibitors of the MDM2-p53 interaction. The present compounds provide stable, formulatable entities that in vivo can lead to potent and selective oral anticancer agents.

### DETAILED DESCRIPTION OF THE INVENTION

The present invention relates to spiroindolinones of the formula

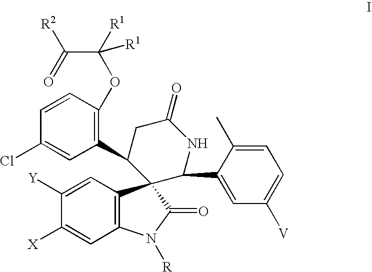

wherein

### X is Cl or Br

### Y is H or F

### V is F or Cl

R¹ is selected from Me, Et or nPr
R² is selected from OH, OMe or NHSO₂Me
R is selected from C(═O)R′, CH₂OH, CH₂OR′, or CH₂C(═O)R′
R′ is selected from lower alkyl, substituted lower alkyl, lower alkoxy, substituted lower alkoxy, cycloalkyl, substituted cycloalkyl, cycloalkoxy or substituted cycloalkoxy.
or a pharmaceutically acceptable salt, ester or enantiomer thereof.

The compounds of formula I are prodrugs of compounds of the formula

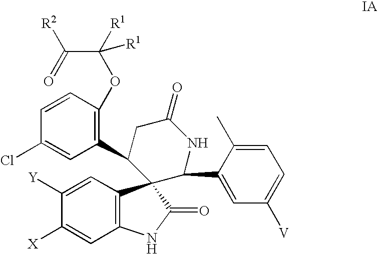

wherein the values for V, X, Y, R¹ are as above and R² is OH or NHSO₂Me. The compounds of formula IA are active as anticancer agents and are claimed in U.S. patent application Ser. Nos. 11/846,597 filed Aug. 29, 2007 and 12/273,035 filed Nov. 18, 2008.

Preferred are compounds of formula I wherein

### X is Cl,

### Y is H,

### V is F or Cl,

### R¹ is Me or Et,

R² is selected from OH, OMe or NHSO₂Me

### R is C(═O)R′ and

R′ is selected from lower alkyl, substituted lower alkyl, lower alkoxy, substituted lower alkoxy, cycloalkyl, substituted cycloalkyl, cycloalkoxy or substituted cycloalkoxy.

Most preferred compounds are those of the formula:
chiral (2′S,3S,4′R)-1-acetyl-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-6-chloro-4′-[5-chloro-2-(1-methoxycarbonyl-1-methyl-ethoxy)-phenyl]-2,3-dihydro-2′-(5-fluoro-2-methyl-phenyl)-2,6′-dioxo spiro[indole-3,3′-piperidine]-1-carboxylic acid tent-butyl ester;
racemic (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
racemic (2′S,3S,4′R)-6-chloro-4′-[5-chloro-2-(1-methoxycarbonyl-1-methyl-ethoxy)-phenyl]-2′-(5-fluoro-2-methyl-phenyl)-1-hydroxymethyl spiro[3H-indole-3,3′-piperidine]-2,6′(1H)-dione;
racemic (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione;
racemic (2′S,3S,4′R)-6-chloro-4′-[5-chloro-2-(1-hydroxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione;
chiral (2′R,3R,4′S)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione;
chiral (2′S,3S,4′R)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione;
racemic (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione;
chiral (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione;
chiral (2′R,3R,4′S)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione;
racemic (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′R,3R,4′S)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
racemic (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione;
chiral (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione;
chiral (2′R,3R,4′S)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione;
racemic (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′R,3R,4′S)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-(2-ethyl-butyryl)-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isobutyryl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
chiral (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
chiral (2′R,3R,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isopropoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2-methoxy-ethoxycarbonyl) spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isobutoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-hexyloxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2,2-dimethyl-propoxycarbonyl) spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-(2-fluoro-ethoxycarbonyl)-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-ethoxycarbonyl-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-methoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-2,6′-dioxo-2,3-dihydro spiro[indole-3,3′-piperidine]-1-carboxylic acid tert-butyl ester and
racemic (2′S,3S,4′R)-1-acetyl-6-chloro-4′-[5-chloro-2-(1-ethyl-1-methanesulfonylaminocarbonyl-propoxy)-phenyl]-5-fluoro-2′-(5-fluoro-2-methyl-phenyl) spiro[3H-indole-3,3′-piperidine]-2,6′(1H)-dione.

In the specification where indicated the various groups may be substituted by 1-5 or, preferably, 1-3 substituents independently selected from the group consisting of lower alkyl, lower-alkenyl, lower-alkynyl, dioxo-lower-alkylene (forming e.g. a benzodioxyl group), halogen, hydroxy, CN, CF₃, NH₂, N(H, lower-alkyl), N(lower-alkyl)₂, aminocarbonyl, carboxy, NO₂, lower-alkoxy, thio-lower-alkoxy, lower-alkylsulfonyl, aminosulfonyl, lower-alkylcarbonyl, lower-alkylcarbonyloxy, lower-alkoxycarbonyl, lower-alkyl-carbonyl-NH, fluoro-lower-alkyl, fluoro-lower-alkoxy, lower-alkoxy-carbonyl-lower-alkoxy, carboxy-lower-alkoxy, carbamoyl-lower-alkoxy, hydroxy-lower-alkoxy, NH₂-lower-alkoxy, N(H, lower-alkyl)-lower-alkoxy, N(lower-alkyl)₂-lower-alkoxy, benzyloxy-lower-alkoxy, mono- or di-lower alkyl substituted amino-sulfonyl and lower-alkyl which can optionally be substituted with halogen, hydroxy, NH₂, N(H, lower-alkyl) or N(lower-alkyl)₂. Preferred substituents for the aryl, heteroaryl and heterocycle rings are halogen, lower alkoxy, lower alkyl and amino.

If alkyl, alkenyl, alkynyl or similar groups are linked with both ends to the same moiety, cyclic structures may result, where two hydrogens of said moiety are being replaced by the two ends of the alkyl, alkenyl, alkynyl or similar group, thus creating cyclic structures, such as, tetralin, macrocycles or spiro compounds.

The term “alkyl” refers to straight- or branched-chain saturated hydrocarbon groups having from 1 to about 20 carbon atoms. In certain embodiments, alkyl substituents may be lower alkyl substituents. The term “lower alkyl” refers to alkyl groups having from 1 to 8 carbon atoms, and in certain embodiments from 1 to 4 carbon atoms. Examples of alkyl groups include, but are not limited to, methyl, ethyl, n-propyl, i-propyl, n-butyl, s-butyl, t-butyl, n-pentyl, and s-pentyl.

As used herein, “cycloalkyl” is intended to refer to any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “alkenyl” as used herein means an unsaturated straight-chain or branched aliphatic hydrocarbon group containing one double bond and having 2 to 8, preferably 2 to 6 carbon atoms. Examples of such “alkenyl group” are vinyl (ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “halogen” as used in the definitions means fluorine, chlorine, iodine or bromine, preferably fluorine and chlorine.

“Aryl” means a monovalent, monocyclic or bicyclic, aromatic carbocyclic hydrocarbon radical, preferably a 6-10 member aromatic ring system. Preferred aryl groups include, but are not limited to, phenyl, naphthyl, tolyl, and xylyl.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole and tetrazolyl.

In the case of aryl or heteroaryl which are bicyclic it should be understood that one ring may be aryl while the other is heteroaryl and both being substituted or unsubstituted.

“Heterocycle” means a substituted or unsubstituted 5 to 8 membered, mono- or bicyclic, aromatic or non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like.

“Hetero atom” means an atom selected from N, O and S.

“Alkoxy, alkoxyl or lower alkoxy” refers to any of the above lower alkyl groups attached to an oxygen atom. Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (6th Ed. 1995) at pp. 196 and 1456-1457.

The compounds of formula I as well as their salts have at least one asymmetric carbon atom and therefore may be present as racemic mixtures or different stereoisomers. The various isomers can be isolated by known separation methods, e.g., chromatography. The invention includes all stereoisomers.

The compounds of the present invention are useful in the treatment or control of cell proliferative disorders, in particular oncological disorders. These compounds and formulations containing said compounds may be useful in the treatment or control of solid tumors, such as, for example, breast, colon, lung and prostate tumors.

A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I or II or III compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

“Effective amount” means an amount that is effective to prevent, alleviate or ameliorate symptoms of disease or prolong the survival of the subject being treated.

“IC₅₀” refers to the concentration of a particular compound required to inhibit 50% of a specific measured activity. IC₅₀ can be measured, inter alia, as is described subsequently.

“Pharmaceutically acceptable ester” refers to a conventionally esterified compound of formula I having a carboxyl group or hydroxy group, which esters retain the biological effectiveness and properties of the compound of formula I and are cleaved in vivo (in the organism) to the corresponding active carboxylic acid or alcohol respectively.

### Synthesis

Compounds of this invention in formula I can be synthesized according to the following general schemes. It will be readily apparent to those of ordinary skill in the art that compounds in formula I can be prepared by substitution of the reagents or agents in the general synthesis routes. Using purification by chiral chromatography, compounds in formula I can be obtained as an optically pure or enriched enantiomers.

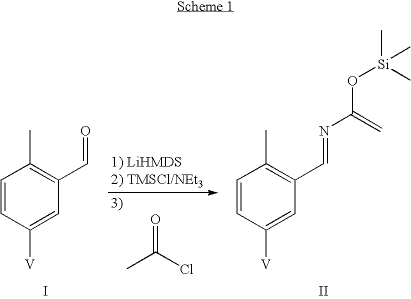

In general an appropriately selected aldehyde I can be reacted with lithium hexamethyldisilamide, chlorotrialkylsilane and acteyl chloride in a one-pot, multi-steps manner to generate 2-aza-1,3-butadiene II (Scheme I) and can be used as a crude product. Ghosez, L. and others have reported the preparation of 2-aza-1,3-butadienes and their use in aza Diels-Alder reaction to form heterocycle (Ref: Tetrahedron 1995, 11021; J. Am. Chem. Soc. 1999, 2617; and literatures cited therein). The appropriately selected aldehyde I are either commercially available or can be synthesized by well-established multiple literature methods.

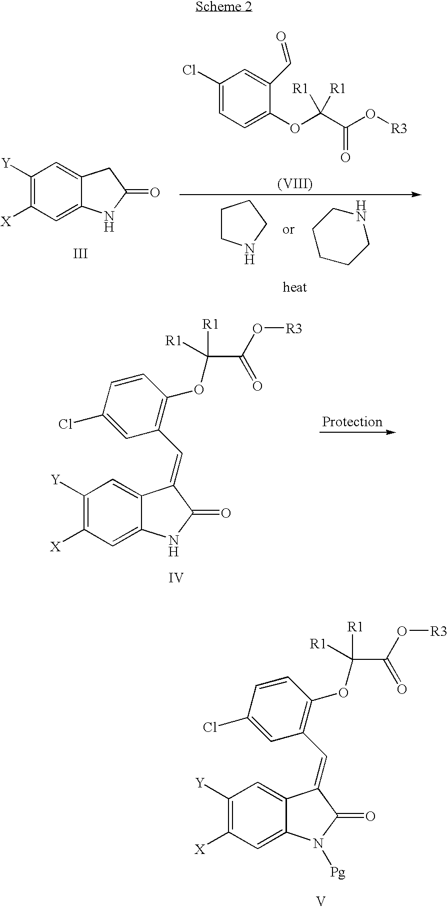

R1 is Me or Et or nPr, and R3 is lower alkyl

Oxindole III can be reacted with an appropriately substituted aldehyde VIII in the presence of base under heated condition in either a protic like methanol, ethanol or an aprotic solvent like toluene, o-xylene to give intermediate IV. The commonly used base is either pyrrolidine or piperidine. Intermediate IV can be protected to give intermediate V. The protective group can be attached by using ethyl chloroformate, di-tert-butyl dicarbonate, SEM-Cl, benzyl bromide, and a base like 4-(dimethylamine)pyridine (DMAP), triethylamine, NaH, or LiH according to well established literature procedures. Examples of protective group formation and their deprotection have been described and reviewed comprehensively by Greene, T. W. et al in “Protective Groups in Organic Synthesis, 2^{nd} Edition. John Wiley & Sons Inc.

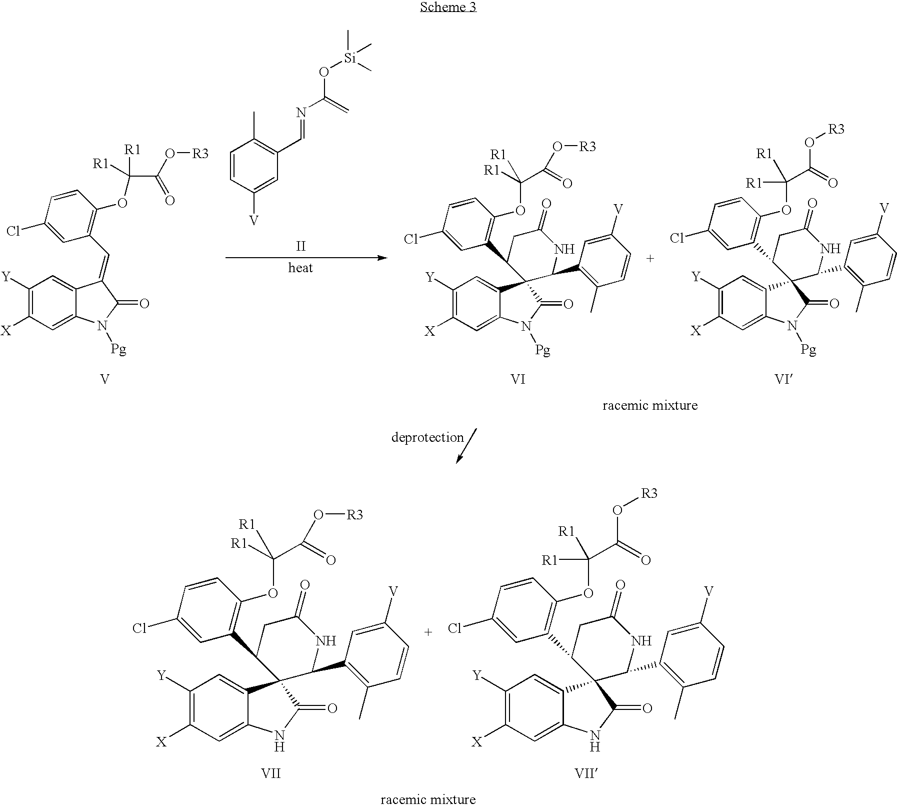

R1 is Me or Et or nPr, and R3 is lower alkyl

Intermediate V can be reacted with a selected 2-aza-butadiene II prepared in Scheme 1 in toluene or o-xylene under heating from 110° C. to 160° C. and anhydrous condition to form intermediate VI and VI′ as the major products shown as a racemic mixture of two enantiomers. A subsequent reaction to remove protective group (Pg) leads to various R₂ derivatized compound VII and VII′. (Scheme 3). In the case Pg is Boc group, Boc group can be removed by either trifluoroacetic acid or prolonged heating at a temperature between 110 to 116° C. during Aza Diels-Alder reaction between V and II without trifluoroacetic acid. Racemic mixture of VI and VI′ or VII and VII′ can be readily resolved into two chiral enantiomers by chiral Super Fluid Chromatography (SFC) or chiral HPLC or chiral column chromatography.

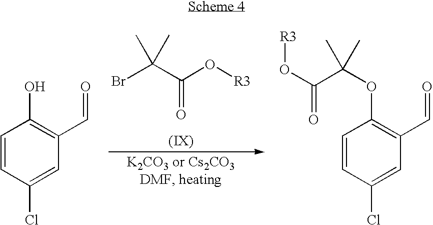

When R1 is methyl, intermediate VIII in Scheme 2 can be prepared by treatment of 5-chlorosalicylaldehyde, and a commercially available reagent IX, a base like K₂CO₃ or Cs₂CO₃ in anhydrous N,N-dimethylformamide under heating conditions. (Scheme 4). When R1 is ethyl or n-propyl, intermediate VIII in Scheme 2 can be prepared in a synthetic route illustrated in Scheme 5.

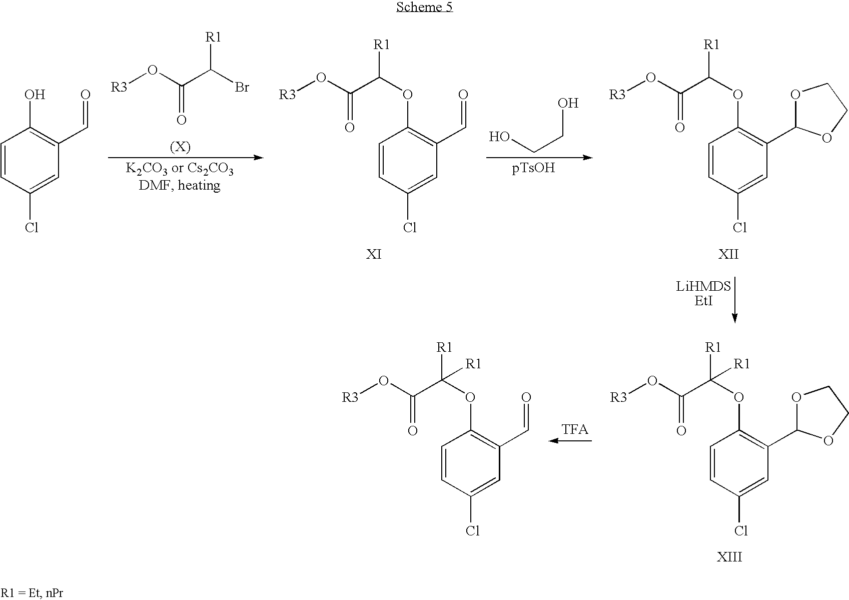

When R2 is NHSO₂Me or OMe in formula I, the corresponding analogues XVI and XVII are prepared according to the methods illustrated in Scheme 6. Compound VII is hydrolyzed to acid XV, followed by a coupling reaction using well-known methods to afford analogues XVI or XVII. Finally, XVI and XVII can be converted into their corresponding prodrug XVIII or XIX by selectly acylation of N1 position under controlled conditions.

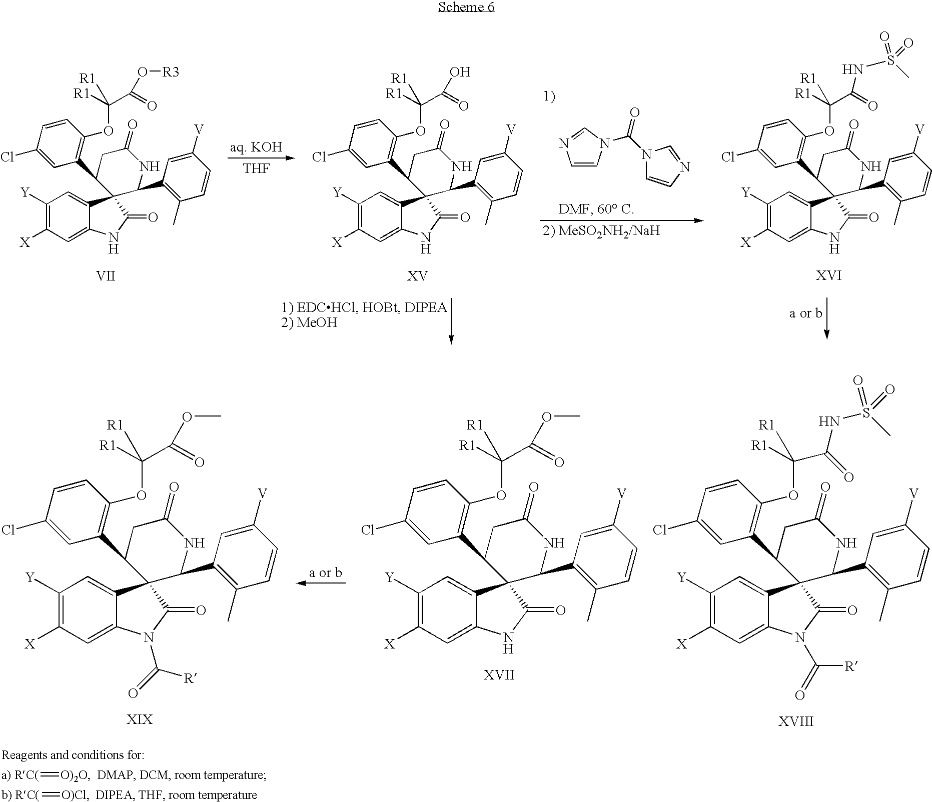

The following examples and references are provided to aid the understanding of the present invention, the true scope of which is set forth in the appended claims.

### Example 1

### Preparation of Intermediate 2-(4-chloro-2-formyl-phenoxy)-2-methyl-propionic acid ethyl ester

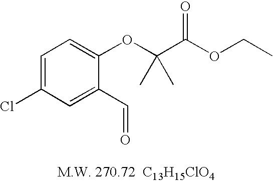

5-Chloro-2-hydroxy-benzaldehyde (7 g, 45 mmol), 2-bromo-2-methyl-propionic acid ethyl ester (11.4 g, 58 mmol), K₂CO₃ (18.6 g, 135 mmol) and KI (0.97 g, 5.8 mmol) were mixed in DMF (20 mL). Then the reaction mixture was heated at 110° C. for 3 h. The mixture was filtered and the filtrate was concentrated. The residue was dissolved in ethyl acetate and washed with 1N NaOH. Then the organic layer was separated, dried over Na₂SO₄ and concentrated to give title compound (7 g)

### Example 2

### Preparation of Intermediate E/Z-2-{4-chloro-2-[6-chloro-2-oxo-1,2-dihydro-indol-ylidenemethyl]-phenoxy}-2-methyl-propionic acid ethyl ester

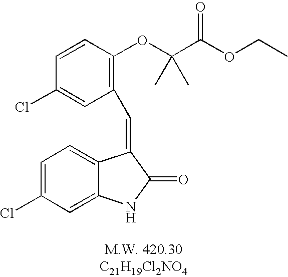

2-(4-chloro-2-formyl-phenoxy)-2-methyl-propionic acid ethyl ester (7 g, 26 mmol) and 6-chlorooxindole (3.6 g, 22 mmol) were mixed in anhydrous methanol (30 mL) at room temperature. Then pyrrolidine (1.85 g, 26 mmol) was added slowly. The reaction mixture was heated at 70° C. for 3 h. Then the mixture was cooled to room temperature and filtered. The precipitate was dried and collected to give E/Z-2-[4-chloro-2-(6-chloro-2-oxo-1,2-dihydro-indol-3-ylidenemethyl)-phenoxy]-2-methyl-propionic acid ethyl ester as a yellow solid (7.2 g).

### Example 3

### Preparation of Intermediate E/Z 6-chloro-3-[5-chloro-2-(1-ethoxycarbonyl-1-methyl-ethoxy)-benzylidene]-2-oxo-2,3-dihydro-indole-1-carboxylic acid tert-butyl ester

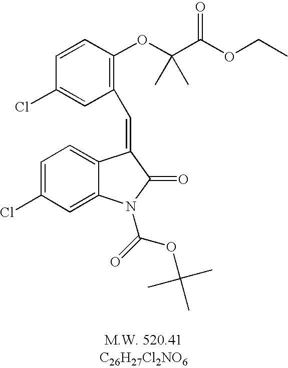

To a solution of E/Z 2-[4-chloro-2-(6-chloro-2-oxo-1,2-dihydro-indol-3-ylidenemethyl)-phenoxy]-2-methyl-propionic acid ethyl ester (7.2 g, 17.2 mmol) in dichloromethane (50 mL) at r.t was added di-tert-butyl-dicarbonate (4.5 g, 20.6 mmol), followed by the addition of 4-dimethylaminopyridine (0.2 g, 1.72 mmol). The reaction mixture was stirred at r.t. for 0.5 h, then the mixture was washed with 0.5N HCl aqueous solution. The organic layer was separated, dried and concentrated to give give title compound as a yellow solid (8 g).

### Example 4

### Preparation of Intermediate 1-(5-fluoro-2-methylphenyl)-3-trimethylsilyoxy-2-aza-1,3-butadiene

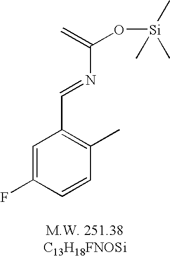

To 1,1,1,3,3,3-hexamethyldisilazane (2.18 mL, 10.5 mmol) (Aldrich) under nitrogen at room temperature was added n-butyllithium (2.5 M, 4.2 mL, 10.5 mmol) (Aldrich). The reaction mixture was stirred at room temperature for 10 minutes. Then dry tetrahydrofuran (30 mL) was added, followed by the addition of 5-fluoro-2-methyl-benzaldehyde (1.38 g, 10 mmol) (Platte). After the mixture was stirred at room temperature for 0.5 h, trimethylsilyl chloride (1.33 mL, 10.5 mmol) (Aldrich) was added dropwise. Then the temperature of the mixture was lowered to 0° C. on a cooling ice bath. To this mixture was added triethylamine (1.9 mL, 13.6 mmol) in one portion, followed by the dropwise addition of a solution of acetyl chloride (0.97 mL, 13.6 mmol) in diethyl ether (50 mL). The cooling bath was removed, and the mixture was stirred at room temperature for 1 h. The mixture was quickly filtered on celite under nitrogen, and filtrate was concentrated under reduced pressure to give crude 1-(5-fluoro-2-methylphenyl)-3-trimethylsilyoxy-2-aza-1,3-butadiene as a yellow gum and used for the next step without further purification.

### Example 5

### Preparation of Intermediate racemic (2′S,3S,4′R)-6-chloro-4′-[5-chloro-2-(1-ethoxycarbonyl-1-methyl-ethoxy)-phenyl]-2′-(5-fluoro-2-methyl-phenyl) spiro[3H-indole-3,3′-piperidine]-2,6′(1H)-dione

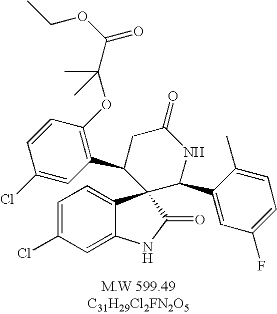

To a toluene solution (50 mL) of 1-(5-fluoro-2-methyl-phenyl)-3-trimethylsilyoxy-2-aza-1,3-butadiene (77 mmol) was added E/Z 6-chloro-3-[5-chloro-2-(1-ethoxycarbonyl-1-methyl-ethoxy)-benzylidene]-2-oxo-2,3-dihydro-indole-1-carboxylic acid tert-butyl ester (8 g, 15.44 mmol). Then the reaction mixture were heated at 130° C. for 2. After the solution was cooled to room temperature, methanol was added, and then the mixture was concentrated. Then a mixture of trifluoroacetic acid (10 mL) and dichloromethane (30 mL) was added. The reaction mixture was stirred at room temperature for 10 min. The solution was concentrated and the residue was purified by Prep-HPLC to give give title compound as a white solid (2.7 g).

m/z (M+H)⁺: 599

### Example 6

### Preparation of Intermediate racemic (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione

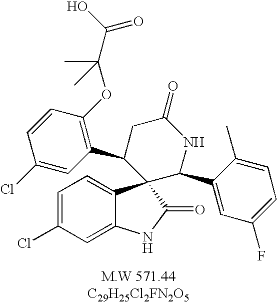

Racemic (2′S,3S,4′R)-6-chloro-4′-[5-chloro-2-(1-ethoxycarbonyl-1-methyl-ethoxy)-phenyl]-2′-(5-fluoro-2-methyl phenyl) spiro[3H-indole-3,3′-piperidine]-2,6′(1H)-dione (2.7 g, 4.5 mmol) was dissolved in THF (20 mL). Then aqueous solution (10 mL) of KOH (0.5 g) was added. The mixture was refluxed for 1 h. After cooled to room temperature, the solution was concentrated and then the residue was acidified to “pH” 2-3 by addition of concentrated aqueous HCl solution. The white solid was collected by filtration to give title compound (1.6 g).

m/z (M+H)⁺: 571

### Example 7

### Preparation of Intermediate chiral (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione

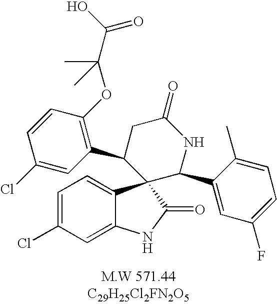

Separation of the two enantiomers from racemic (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione was conducted by chiral HPLC to provide chiral (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione as a white solid (8 mg) and chiral (2′R, 3R,4′S)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione as a white solid (8 mg).

m/z (M+H)⁺: 571

### Example 8

### Preparation of chiral (2′S,3S,4′R)-1-acetyl-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione

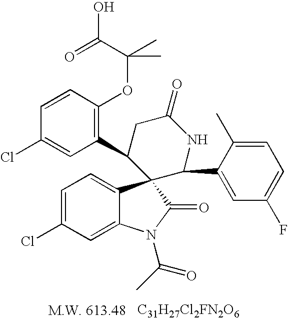

At room temperature, a mixture of chiral (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione (300 mg, 0.526 mmol), acetic anhydride (107 mg, 1.053 mmol) and Et₃N (159 mg, 1.579 mmol) in THF (5 mL) was stirred for 1 h. After the solution was concentrated, the residue was purified by Prep-HPLC to give the title compound as a white solid (138 mg).

### Example 9

### Preparation of Intermediate racemic (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

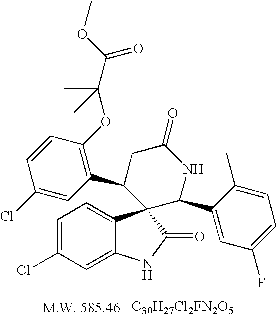

To a mixture of racemic (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione (0.5 g, 0.88 mmol) prepared in Example 6, EDC.HCl (0.33 g, 1.73 mmol), HOBt (0.24 g, 1.78 mmol) and DIPEA (0.46 mL, 2.64 mmol) in THF (3 mL) was added methanol (56 mg, 1.75 mmol). After the mixture was stirred at room temperature for two days, it was concentrated and the residue was purified by flash column to give the title compound as a white solid (450 mg).

m/z (M+H)⁺: 585

### Example 10

### Preparation of Intermediate chiral (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

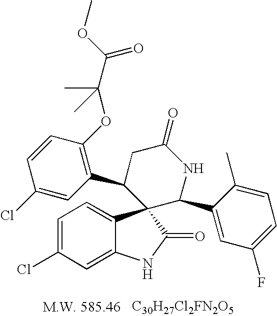

Separation of the two enantiomers from racemic (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (450 mg) was conducted by chiral SFC to provide chiral (2′S,3S, 4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione as a white solid (165 mg).

m/z (M+H)⁺: 585

### Example 11

### Preparation of chiral (2′S,3S,4′R)-6-chloro-4′-[5-chloro-2-(1-methoxycarbonyl-1-methyl-ethoxy)-phenyl]-2,3-dihydro-2′-(5-fluoro-2-methyl-phenyl)-2,6′-dioxo spiro[indole-3,3′-piperidine]-1-carboxylic acid tent-butyl ester

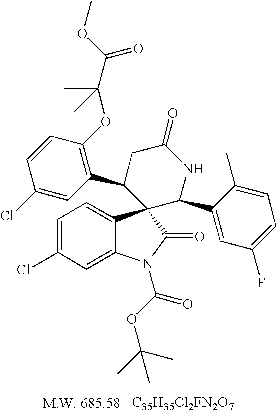

At room temperature, to a solution of chiral (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (250 mg, 0.428 mmol) in dichloromethane (2 mL) was added di-tert-butyl-dicarbonate (103 mg, 0.47 mmol), followed by the addition of 4-dimethylaminopyridine (5 mg, 0.041 mmol). The reaction mixture was stirred at room temperature for 1 h. Then the mixture was concentrated and the residue was purified by chromatography to give the title compound as a white solid (205 mg).

m/z (M+H)⁺: 685

### Example 12

### Preparation of racemic (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

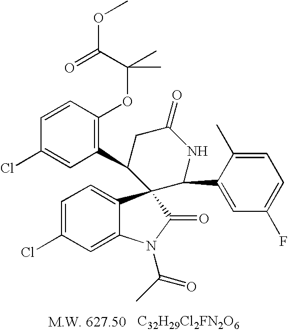

At 0° C., a mixture of racemic (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (1 g, 1.71 mmol) prepared in Example 9, acetic anhydride (0.174 g, 1.71 mmol) and DMAP (0.02 g, 0.171 mmol) in CH₂Cl₂ (15 mL) was stirred for 1 h. Then the solution was washed with water twice, dried and concentrated. The residue was washed with methanol twice to give the title compound as a white solid (800 mg).

### Example 13

### Preparation of chiral (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

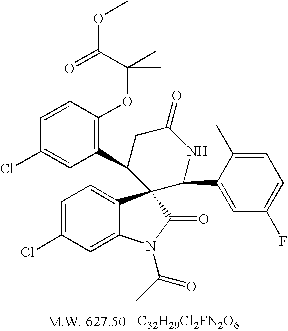

Separation of the two enantiomers from racemic (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione was conducted by chiral SFC to provide chiral (2′S,3S,4′R)-6-1-acetyl-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione as a white solid (143 mg).

m/z (M+H)⁺: 627

### Example 14

### Preparation of Intermediate E/Z-2-{4-chloro-2-[6-chloro-2-oxo-1-(2-trimethylsilanyl-ethoxymethyl)-1,2-dihydro-indol-3-ylidenemethyl]-phenoxy}-2-methyl-propionic acid methyl ester

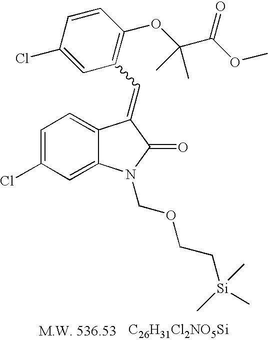

To a solution of E/Z-2-[4-Chloro-2-(6-chloro-2-oxo-1,2-dihydro-indol-3-ylidenemethyl)-phenoxy]-2-methyl-propionic acid methyl ester (14.2 g, 35.06 mmol) in N,N-dimethylformamide (100 mL) at 0° C. was added NaH (60% in mineral oil) (1.4 g, 35.06 mmol) (Aldrich), followed by the dropwise addition of 2-(trimethylsilyl)ethoxymethyl chloride (5.84 g, 35.06 mmol) in tetrahydrofuran (70 mL). The reaction mixture was stirred at 0° C. for 2 h, then poured into ice-water. The aqueous solution was extracted with ethyl acetate twice. The combined organic phases were dried over anhydrous Na₂SO₄, concentrated and the residue was purified by chromatography to give the title compound as a yellow oil (14 g).

### Example 15

### Preparation of Intermediate racemic (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione

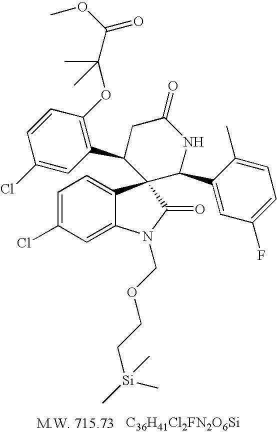

### Method a.

To a solution of 1-(5-fluoro-2-methylphenyl)-3-trimethylsilyoxy-2-aza-1,3-butadiene (130 mmol) prepared in Example 4 in toluene (130 mL) was added E/Z-2-{4-chloro-2-[6-chloro-2-oxo-1-(2-trimethylsilanyl-ethoxymethyl)-1,2-dihydro-indol-3-ylidenemethyl]-phenoxy}-2-methyl-propionic acid methyl ester (14 g, 26.2 mmol). Under nitrogen the reaction mixture was stirred at 70° C. overnight. After cooled to room temperature, methanol (100 mL) was added. Then the mixture was concentrated and the residue was purified by chromatography to give the title compound as an yellow solid (2.4 g).

### Method b.

At 0° C., to a solution of racemic (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (2.1 g, 3.59 mmol) prepared in Example 9 in anhydrous DMF (30 mL) was added NaH (60% in mineral oil) (0.158 g, 1.88 mmol) (Aldrich), followed by the dropwise addition of 2-(trimethylsilyl)ethoxymethyl chloride (0.6 g, 3.59 mmol) in tetrahydrofuran (10 mL). After stirred at 0° C. for 2 h, the reaction mixture was poured into water. Then the aqueous phase was extracted with EtOAc thrice and the combined organic layers were dried, concentrated to give the crude product.

### Example 16

### Preparation of racemic (2′S,3S,4′R)-6-chloro-4′-[5-chloro-2-(1-methoxycarbonyl-1-methyl-ethoxy)-phenyl]-2′-(5-fluoro-2-methyl-phenyl)-1-hydroxymethyl spiro[3H-indole-3,3′-piperidine]-2,6′(1H)-dione

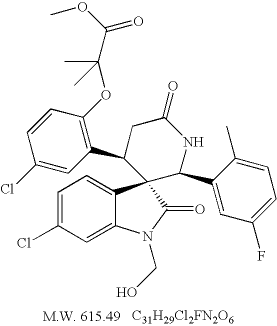

To a solution of racemic (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione (1.4 g, 1.96 mmol) in dichloromethane (20 mL) was added trifluoracetic acid (10 mL). After stirred for 0.5 h at room temperature, the reaction solution was concentrated. The residue was diluted with ethyl acetate, washed with saturated sodium bicarbonate solution, brine and concentrated. The residue was used for the next step reaction directly without further purification.

### Example 17

### Preparation of racemic (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

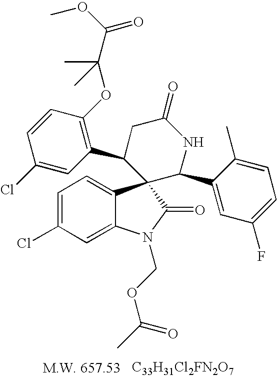

To the solution of crude racemic (2′S,3S,4′R)-6-chloro-4′-[5-chloro-2-(1-methoxycarbonyl-1-methyl-ethoxy)-phenyl]-2′-(5-fluoro-2-methyl-phenyl)-1-hydroxymethyl spiro[3H-indole-3,3′-piperidine]-2,6′(1H)-dione (1.2 g, 1.96 mmol) in CH₂Cl₂ (10 mL) was added acetic anhydride (200 mg, 1.96 mmol) and Et₃N (396 mg, 3.92 mmol). After stirred for 0.5 h, the reaction mixture was concentrated and the residue was purified by Prep-HPLC to give the title compound as a white solid (700 mg).

### Example 18

### Preparation of chiral (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione

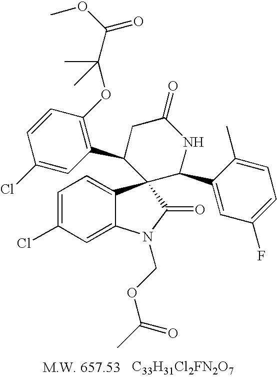

Separation of the two enantiomers from racemic (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (700 mg) was conducted by chiral SFC to provide chiral (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione as a white solid (205 mg).

m/z (M+H)⁺: 657

### Example 19

### Preparation of chiral (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione

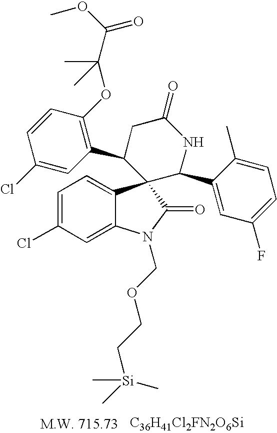

At 0° C., to a solution of chiral (2′S,3S,4′R)-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione (400 mg, 0.684 mmol) prepared in Example 10 in anhydrous DMF (5 mL) was added NaH (60% in mineral oil) (30 mg, 0.753 mmol) (Aldrich), followed by the dropwise addition of 2-(trimethylsilyl)ethoxymethyl chloride (114 mg, 0.684 mmol) in tetrahydrofuran (5 mL). After stirred at 0° C. for 2 h, the mixture was concentrated and the residue was purified by Prep-HPLC to give the title compound as a white solid (184 mg).

### Example 20

### Preparation of racemic (2′S,3S,4′R)-6-chloro-4′-[5-chloro-2-(1-hydroxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione

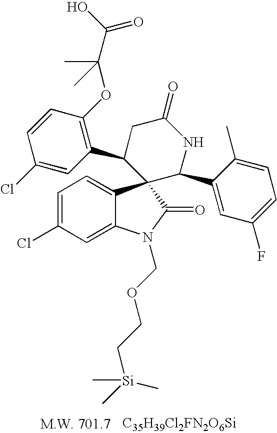

A mixture of the crude racemic (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione (0.6 g) prepared in Example 15, NaOH (287 mg, 7.2 mmol), H₂O (5 mL) and methanol (5 mL) was heated at 80° C. for 1 h. Then methanol was removed by vacuum and the aqueous solution was acidified by concentrated hydrochloride acid to “pH” 1-2. The yellow precipitate was collected by filtration and purified by Prep-HPLC to give the title compound as a white solid (300 mg).

m/z (M+H)⁺: 701

### Example 21

### Preparation of Intermediate racemic (2′S,3S,4′R)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione

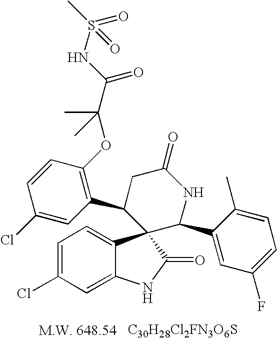

A solution of racemic (2′S,3S,4′R)-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione (16 g, 0.028 mol) prepared in Example 6 and CDI (9 g, 0.056 mol) in DMF (70 mL) was heated at 65° C. for 2 h. Then to this solution was added a mixture of methanesulfonamide (16 g, 0.168 mol) and NaH (5.6 g, 60%, 0.14 mol) in DMF (100 mL), which had been stirred for 2 h at room temperature. After the resulting mixture was stirred at room temperature for 2 h, it was poured into water and the aqueous solution was acidified to “pH” 1-2 by addition of concentrated hydrochloric acid. After the aqueous phase was extracted with EtOAc twice, the combined organic phases were dried over anhydrous Na₂SO₄, concentrated and the residue was purified by recrystallized to give the title compound (11.4 g).

m/z (M+H)⁺: 648

### Example 22

### Preparation of Intermediate chiral (2′S,3S,4′R)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione

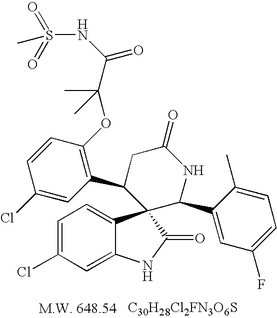

Separation of the two enantiomers from racemic (2′S,3S,4′R)-6-chloro-4′-[5-chloro-2-(2-methane sulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione (50 mg), was conducted by chiral SFC to provide chiral (2′S,3S,4′R)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione as a white solid (10 mg).

m/z (M+H)⁺: 648

### Example 23

### Preparation of Intermediate chiral (2′R,3R,4′S)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione

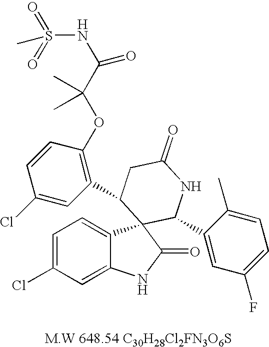

In the SFC chiral separation of the two enantiomers from racemic (2′S,3S,4′R)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione (50 mg) in Example 22, chiral (2′R,3R,4′S)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione was obtained as a white solid (15 mg).

m/z (M+H)⁺: 648

### Example 24

### Preparation of chiral (2′R,3R,4′S)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione

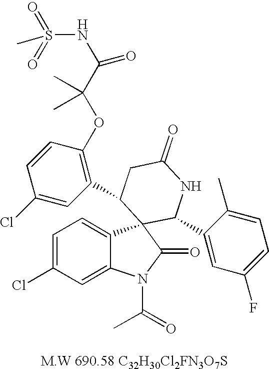

At room temperature, to a mixture of chiral (2′R,3R,4′S)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione (300 mg, 0.46 mmol) prepared in Example 23 and acetic anhydride (75 mg, 0.74 mmol) in DCM (5 mL) was added DMAP (100 mg, 0.82 mmol) slowly. After stirred for 1 h, the solution was washed by 0.5N HCl solution twice, dried over anhydrous Na₂SO₄ and concentrated to give the crude product. The crude product was washed with diethyl ether to give the title compound (200 mg).

m/z (M+H)⁺: 690

### Example 25

### Preparation of chiral (2′S,3S,4′R)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione

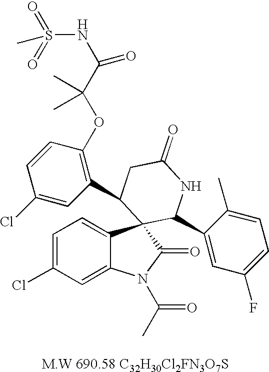

At room temperature, to a mixture of chiral (2′S,3S,4′R)-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione (406 mg, 0.63 mmol) prepared in Example 22 and acetic anhydride (102 mg, 1 mmol) in DCM (8 mL) was added DMAP (100 mg, 0.82 mmol) slowly. After stirred for 1 h, the solution was washed by 0.5N HCl solution twice, dried over anhydrous Na₂SO₄ and concentrated to give the crude product. The crude product was washed with diethyl ether to give the title compound (300 mg).

m/z (M+H)⁺: 690.

### Example 26

### Preparation of Intermediate 2-(4-chloro-2-formyl-phenoxy)-butyric acid methyl ester

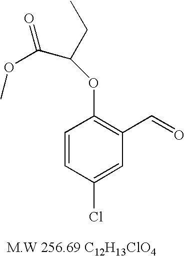

A mixture of 5-chloro-2-hydroxy-benzaldehyde (156 g, 1 mol), 2-bromo-butyric acid methyl ester (271 g, 1.5 mol), KI (2 g, 0.012 mol) and K₂CO₃ (276 g, 2 mol) in DMF (500 mL) was heated at 130° C. for 2 h. After cooled to room temperature, the mixture was concentrated. The residue was partitioned between EtOAc and water. The organic layer was washed with water, brine, dried over anhydrous Na₂SO₄ and concentrated to give the title compound (240 g).

### Example 27

### Preparation of Intermediate of 2-(4-chloro-2-[1,3]dioxolan-2-yl-phenoxy)-butyric acid methyl ester

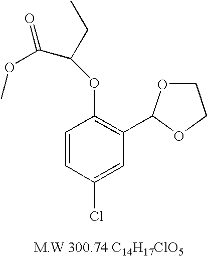

A mixture of 2-(4-chloro-2-formyl-phenoxy)-butyric acid methyl ester (50 g, 0.195 mol), ethylene glycol (89 mL, 1.56 mol) and p-toluenesulfonic acid (2.8 g, 16.5 mmol) in toluene (400 mL) was refluxed with a Dean-Stark trap attached to remove the water. After 3 h, the reaction was cooled and washed with water, saturated NaHCO₃ and water, dried over anhydrous Na₂SO₄ and concentrated to give the title compound as a light yellow oil (40 g).

### Example 28

### Preparation of Intermediate of 2-(4-chloro-2-[1,3]dioxolan-2-yl-phenoxy)-2-ethyl-butyric acid methyl ester

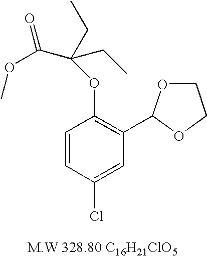

Lithium bis(trimethylsilyl)amide (60 mL, 60 mmol, 1 M in THF) was slowly added to a solution of 2-(4-chloro-2-[1,3]dioxolan-2-yl-phenoxy)-butyric acid methyl ester (15 g, 50 mmol) in anhydrous THF (150 mL) at −78° C. After the mixture was stirred for 15 min, iodoethane (9.3 g, 60 mmol) was added. The mixture was allowed to warm to room temperature and stirred for 2 h. Then the mixture was diluted with ethyl acetate, washed with a saturated aqueous solution of NH₄Cl, dried over anhydrous Na₂SO₄ and concentrated to give the crude product as a oil (16 g).

### Example 29

### Preparation of Intermediate of 2-(4-chloro-2-formyl-phenoxy)-2-ethyl-butyric acid methyl ester

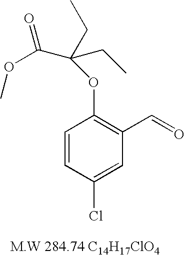

A solution of 2-(4-chloro-2-[1,3]dioxolan-2-yl-phenoxy)-2-ethyl-butyric acid methyl ester (16 g, 48.8 mmol) in trifluoroacetic acid (20 mL) was stirred at room temperature for 3 h. Then the mixture was concentrated and the residue was partitioned between EtOAc and water. The organic layer was washed with 1N NaOH solution, water, dried over anhydrous Na₂SO₄ and concentrated to give the title compound (13 g).

### Example 30

### Preparation of Intermediate of E/Z-2-[4-chloro-2-(6-chloro-2-oxo-1,2-dihydro-indol-3-ylidenemethyl)-phenoxy]-2-ethyl-butyric acid methyl ester

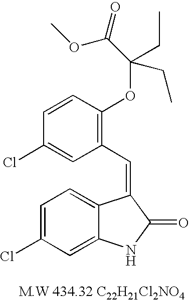

To the mixture of 6-chlorooxindole (8.3 g, 49.7 mmol) and 2-(4-chloro-2-formyl-phenoxy)-2-ethyl-butyric acid methyl ester (13 g, 45.8 mmol) in methanol (200 mL) was added pyrrolidine (4.1 mL, 49.7 mmol) dropwise. The mixture was then heated at 70° C. for 2 h. After cooled to room temperature, the mixture was filtered and the precipitate was collected, dried to give the title compound as a yellow solid (15.5 g).

### Example 31

### Preparation of Intermediate E/Z-3-[5-chloro-2-(1-ethyl-1-methoxycarbonyl-propoxy)-benzylidene]-6-chloro-2-oxo-2,3-dihydro-indole-1-carboxylic acid tert-butyl ester

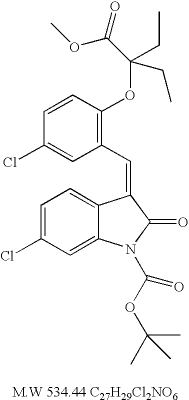

To a solution of E/Z-2-[4-chloro-2-(6-chloro-2-oxo-1,2-dihydro-indol-3-ylidenemethyl)-phenoxy]-2-ethyl-butyric acid methyl ester (15.5 g, 36 mmol) in dichloromethane (200 mL) at room temperature was added di-tert-butyl-dicarbonate (8.6 g, 39 mmol), followed by the addition of 4-dimethylaminopyridine (0.4 g, 3.3 mmol). After the reaction mixture was stirred at room temperature for 1 h, the solution was washed with 1 M HCl and brine twice, dried over anhydrous Na₂SO₄ and concentrated to give the title compound as a yellow solid (15 g).

### Example 32

### Preparation of Intermediate racemic (2′S,3S,4′R)-6-chloro-4′-[5-chloro-2-(1-ethyl-1-methoxycarbonyl-propoxy)-phenyl]-2′-(5-fluoro-2-methyl-phenyl) spiro[3H-indole-3,3′-piperidine]-2,6′(1H)-dione

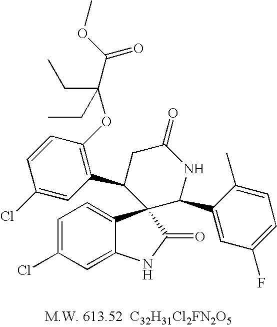

In a manner similar to the method described in Example 5, E/Z-3-[5-chloro-2-(1-ethyl-1-methoxycarbonyl-propoxy)-benzylidene]-6-chloro-2-oxo-2,3-dihydro-indole-1-carboxylic acid tert-butyl ester (7.6 g, 15 mmol) was reacted with 1-(5-fluoro-2-methyl-phenyl)-3-trimethylsilyoxy-2-aza-1,3-butadiene (60 mmol) in toluene to give the title compound as a white solid (2.2 g).

m/z (M+H)⁺: 613

### Example 33

### Preparation of Intermediate racemic (2′S,3S,4′R)-6-chloro-4′-[5-chloro-2-(1-ethyl-1-hydroxycarbonyl-propoxy)-phenyl]-2′-(5-fluoro-2-methyl-phenyl) spiro[3H-indole-3,3′-piperidine]-2,6′(1H)-dione

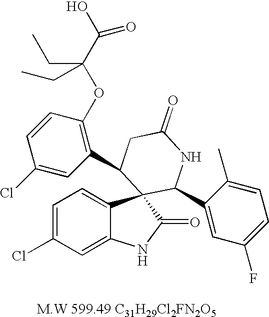

A mixture of racemic (2′S,3S,4′R)-6-chloro-4′-[5-chloro-2-(1-ethyl-1-methoxycarbonyl-propoxy)-phenyl]-2′-(5-fluoro-2-methyl-phenyl) spiro[3H-indole-3,3′-piperidine]-2,6′(1H)-dione (200 mg, 0.33 mmol), LiOH.H₂O (69 mg, 1.16 mmol), H₂O (2 mL) and methanol (20 mL) was refluxed for 2 h. After cooled to room temperature, the solution was concentrated and the residue was acidified to “pH” 2-3 by addition of concentrated HCl solution. The precipitate was collected by filtration to give the title compound as a white solid (57 mg).

m/z (M+H)⁺: 599

### Example 34

### Preparation of Intermediate racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione

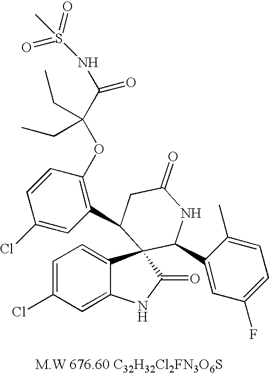

A solution of racemic (2′S,3S,4′R)-6-chloro-4′-[5-chloro-2-(1-ethyl-1-hydroxycarbonyl-propoxy)-phenyl]-2′-(5-fluoro-2-methyl-phenyl) spiro[3H-indole-3,3′-piperidine]-2,6′(1H)-dione (30 mg, 0.05 mmol) and CDI (20 mg, 0.12 mmol) in DMF (1 mL) was heated at 60° C. for 2 h. Then to this solution was added a mixture of methanesulfonamide (28 mg, 0.3 mmol) and NaH (12 mg, 60%, 0.3 mmol) in DMF (1 mL), which had been stirred for 2 h at room temperature. After the resulting mixture was stirred at room temperature for 1 h, it was poured into water and the aqueous solution was acidified to “pH” 1-2 by addition of concentrated HCl solution. The aqueous phase was extracted with EtOAc twice, The combined organic phases were dried over anhydrous Na₂SO₄, concentrated and the residue was purified by flash column to give the title compound as a white solid (10 mg).

m/z (M+H)⁺: 676

### Example 35

### Preparation of racemic (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione

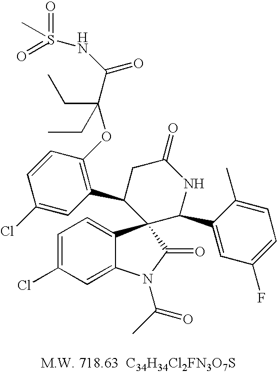

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (540 mg, 0.8 mmol) and acetic anhydride (98 mg, 0.96 mmol) in DCM (20 mL) was added DMAP (10 mg, 0.08 mmol) slowly. After the mixture was stirred for 2 h, the solution was washed by 0.5N HCl solution twice, dried over anhydrous Na₂SO₄ and concentrated. The residue was purified by flash column to give the title compound as a white solid (450 mg).

### Example 36

### Preparation of chiral (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione

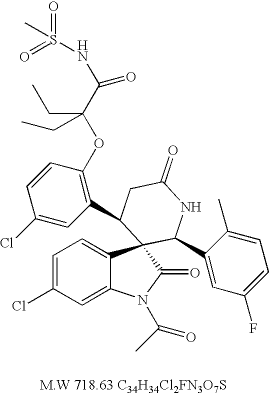

Separation of the two enantiomers from racemic (2′S,3S,4′R)-1-acetyl-6-chloro-4′-[5-chloro-2-(1-ethyl-1-methanesulfonylamino carbonyl-propoxy)-phenyl]-2′-(5-fluoro-2-methyl-phenyl)-spiro[3H-indole-3,3′-piperidine]-2,6′(1H)-dione (50 mg), was conducted by chiral SFC to provide chiral (2′S,3S,4′R)-1-acetyl-6-chloro-4′-[5-chloro-2-(1-ethyl-1-methanesulfonylamino carbonyl-propoxy)-phenyl]-2′-(5-fluoro-2-methyl-phenyl)-spiro[3H-indole-3,3′-piperidine]-2,6′(1H)-dione as a white solid (15 mg).

m/z (M+H)⁺: 718

### Example 37

### Preparation of chiral (2′R,3R,4′S)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione

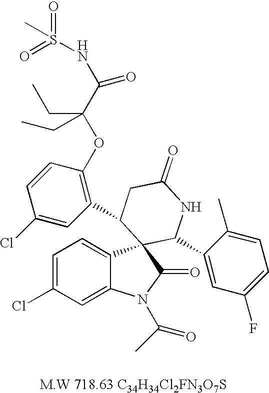

In the SFC chiral separation of the two enantiomers from racemic (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg) in Example 36, chiral (2′R,3R,4′S)-1-acetyl-6-chloro-4′-[5-chloro-2-(1-ethyl-1-methanesulfonylamino carbonyl-propoxy)-phenyl]-2′-(5-fluoro-2-methyl-phenyl)-spiro[3H-indole-3,3′-piperidine]-2,6′(1H)-dione was obtained as a white solid (17 mg).

m/z (M+H)⁺: 718

### Example 38

### Preparation of Intermediate 1-(5-chloro-2-methyl-phenyl)-3-trimethylsilyoxy-2-aza-1,3-butadiene

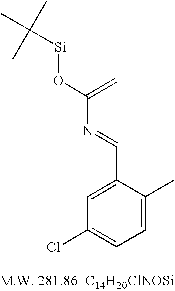

To dry tetrahydrofuran (100 mL) was added 1M THF solution of LiHMDS (97 mmol, 97 mL) under Ar at room temperature, followed by the addition of 5-chloro-2-methyl-benzaldehyde (15 g, 97 mmol). After the mixture was stirred at room temperature for 1 h, trimethylsilyl chloride (12.3 mL, 97 mmol) was added dropwise. Then the temperature of the mixture was lowered to 0° C. on a cooling ice bath. To this mixture was added triethylamine (17.6 mL, 126 mmol) in one portion, followed by the dropwise addition of a solution of acetyl chloride (9 mL, 126 mmol) in diethyl ether (200 mL). The cooling bath was removed, and the mixture was stirred at room temperature overnight. The mixture was quickly filtered on celite under nitrogen, and filtrate was concentrated under reduced pressure to give crude 1-(5-chloro-2-methyl-phenyl)-3-trimethylsilyoxy-2-aza-1,3-butadiene as a yellow gum and used for the next step without further purification.

### Example 39

### Preparation of Intermediate racemic (2′S,3S,4′R)-6-chloro-4′-[5-chloro-2-(1-ethyl-1-methanesulfonylaminocarbonyl-propoxy)-phenyl]-2′-(5-chloro-2-methyl-phenyl) spiro[3H-indole-3,3′-piperidine]-2,6′(1H)-dione

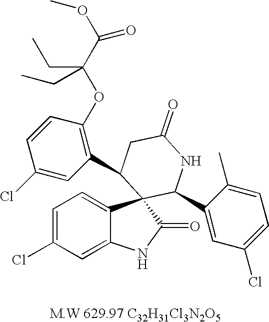

In a manner similar to the method described in Example 5, E/Z-3-[5-chloro-2-(1-ethyl-1-methoxycarbonyl-propoxy)-benzylidene]-6-chloro-2-oxo-2,3-dihydro-indole-1-carboxylic acid tert-butyl ester (2.63 g, 4.9 mmol) prepared in Example 31 was reacted with 1-(5-chloro-2-methylphenyl)-3-trimethylsilyoxy-2-aza-1,3-butadiene (20 mmol) prepared in Example 4 in toluene (20 mL) to give the title compound as a white solid (600 mg).

m/z (M+H)⁺: 629

### Example 40

### Preparation of Intermediate racemic (2′S,3S,4′R)-6-chloro-4′-[5-chloro-2-(1-ethyl-1-hydroxycarbonyl-propoxy)-phenyl]-2′-(5-chloro-2-methyl-phenyl) spiro[3H-indole-3,3′-piperidine]-2,6′(1H)-dione

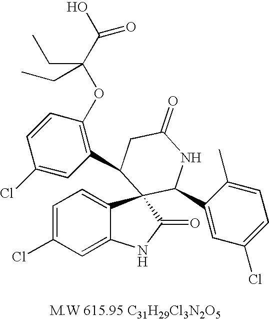

A mixture of racemic (2′S,3S,4′R)-6-chloro-4′-[5-chloro-2-(1-ethyl-1-methoxycarbonyl-propoxy)-phenyl]-2′-(5-chloro-2-methyl-phenyl) spiro[3H-indole-3,3′-piperidine]-2,6′(1H)-dione (100 mg, 0.159 mmol), LiOH (19.87 mg, 0.8 mmol), H₂O (2 mL) and methanol (3 mL) was heated at 40° C. for 4 h. Then methanol was removed by vacuum. The aqueous solution was acidified to “pH” 1-2 by addition of concentrated hydrochloride acid. The precipitate was collected by filtration and purified by Prep-HPLC to give the title compound as a white solid (50 mg).

m/z (M+H)⁺: 615

### Example 41

### Preparation of Intermediate racemic (2′S,3S,4′R)-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

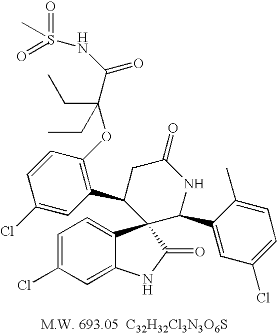

A solution of racemic (2′S,3S,4′R)-6-chloro-4′-[5-chloro-2-(1-ethyl-1-hydroxycarbonyl-propoxy)-phenyl]-2′-(5-chloro-2-methyl-phenyl) spiro[3H-indole-3,3′-piperidine]-2,6′(1H)-dione (150 mg, 0.244 mmol) and CDI (80 mg, 0.49 mmol) in DMF (2 mL) was heated at 60° C. for 2 h. Then to this solution was added a mixture of methanesulfonamide (231 mg, 2.44 mmol) and NaH (78 mg, 60%, 1.95 mmol) in DMF (3 mL), which had been stirred for 2 h at room temperature. After the resulting mixture was stirred at room temperature for 1 h, it was poured into water and the aqueous solution was acidified to “pH” 2-3 by addition of concentrated HCl solution. After the aqueous phase was extracted with EtOAc twice, the combined organic phases were dried over anhydrous Na₂SO₄, concentrated and the residue was purified by flash column to give the title compound as a white solid (10 mg).

m/z (M+H)⁺: 692

### Example 42

### Preparation of racemic (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

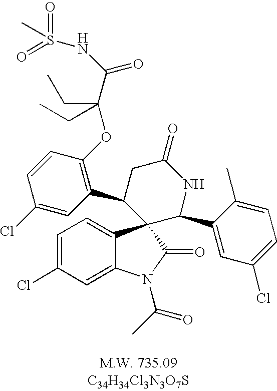

At room temperature, to a mixture of racemic (2′S,3S,4′R)-6-chloro-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione (400 mg, 0.58 mmol) and acetic anhydride (71 mg, 0.69 mmol) in DCM (20 mL) was added DMAP (7 mg, 0.06 mmol) slowly. After the mixture was stirred for 2 h, the solution was washed by 0.5N HCl solution twice, dried over anhydrous Na₂SO₄ and concentrated. The residue was purified by Prep-HPLC to give the title compound as a white solid (100 mg).

### Example 43

### Preparation of chiral (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

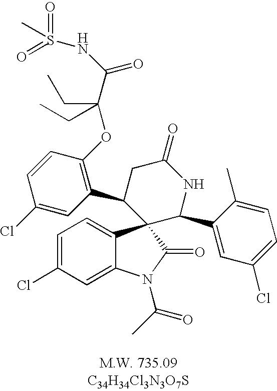

Separation of the two enantiomers from racemic (2′S,3S,4′R)-1-acetyl-6-chloro-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione (50 mg) was conducted by chiral SFC to provide chiral (2′S,3S,4′R)-1-acetyl-6-chloro-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione as a white solid (15 mg) m/z (M+H)⁺: 734

### Example 44

### Preparation of chiral (2′R,3R,4′S)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

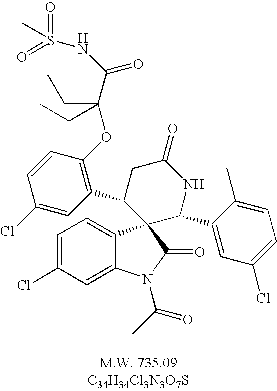

In SFC chiral separation of the two enantiomers from racemic (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione (50 mg) in Example 43, chiral (2′R,3R,4′S)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione was obtained as a white solid (15 mg).

m/z (M+H)⁺: 734

### Example 45

### Preparation of Intermediate 2-(4-chloro-2-formyl-phenoxy)-pentanoic acid ethyl ester

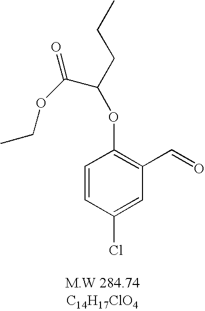

A mixture of 5-chloro-2-hydroxy-benzaldehyde (15 g, 0.1 mol), 2-Bromo-pentanoic acid ethyl ester (27 g, 0.13 mol) and K₂CO₃ (27 g, 0.2 mol) in DMF (100 mL) was heated at 140° C. for 1 h. After cooled to room temperature, the mixture was poured into water and the aqueous phase was extracted with EtOAc thrice. The combined organic phases were washed with water and brine, dried over anhydrous Na₂SO₄ and concentrated. The residue was purified by flash column to give the title compound as a colorless oil (24 g).

### Example 46

### Preparation of Intermediate 2-(4-chloro-2-[1,3]dioxolan-2-yl-phenoxy)-pentanoic acid ethyl ester

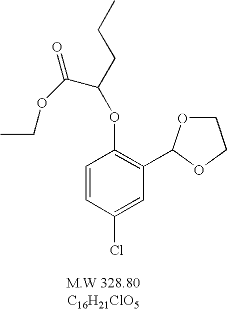

A mixture of 2-(4-chloro-2-formyl-phenoxy)-pentanoic acid ethyl ester (15 g, 53 mmol), ethylene glycol (25 mL, 440 mmol) and p-toluenesulfonic acid (0.8 g, 4.65 mmol) in toluene (150 mL) was refluxed with a Dean-Stark trap attached to remove water. After 3 h, the reaction was cooled and washed with water, saturated NaHCO₃ solution and water, dried over anhydrous Na₂SO₄ and concentrated to give the title compound as a light yellow oil (16 g).

### Example 47

### Preparation of Intermediate 2-(4-chloro-2-[1,3]dioxolan-2-yl-phenoxy)-2-propyl-pentanoic acid ethyl ester

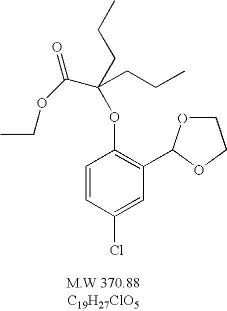

Lithium bis(trimethylsilyl)amide (26 mL, 26 mmol, 1 M in THF) was slowly added to a solution of 2-(4-Chloro-2-[1,3]dioxolan-2-yl-phenoxy)-pentanoic acid ethyl ester (6.6 g, 20 mmol) in 60 mL of anhydrous THF at −78° C. After the mixture was stirred for 30 min at −78° C., 1-Iodopropane (4 mL, 40 mmol) was added. The mixture was allowed to warm to room temperature and stirred for 2 h. Then the mixture was diluted with ethyl acetate, washed with a saturated aqueous solution of NH₄Cl, dried over anhydrous Na₂SO₄ and concentrated to give the title compound as a yellow oil (5 g).

### Example 48

### Preparation of Intermediate 2-(4-chloro-2-formyl-phenoxy)-2-propyl-pentanoic acid ethyl ester

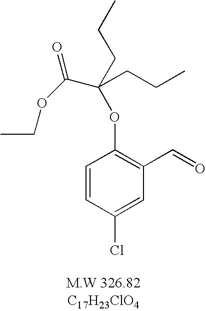

A solution of 2-(4-chloro-2-[1,3]dioxolan-2-yl-phenoxy)-2-propyl-pentanoic acid ethyl ester (15 g, 42 mmol) in TFA (30 mL) was stirred at room temperature overnight. Then the mixture was concentrated and the residue was partitioned between EtOAc and water. The organic phase was washed with 1N NaOH solution, water, dried over anhydrous Na₂SO₄ and concentrated to give the title compound (14 g).

### Example 49

### Preparation of Intermediate E/Z-2-[4-chloro-2-(6-chloro-2-oxo-1,2-dihydro-indol-3-ylidenemethyl)-phenoxy]-2-propyl-pentanoic acid ethyl ester

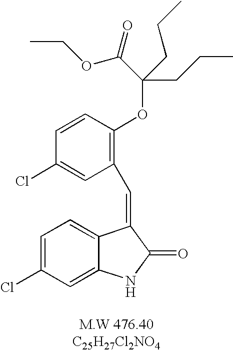

To the mixture of 6-chlorooxindole (9.3 g, 55.7 mmol) and 2-(4-chloro-2-formyl-phenoxy)-2-propyl-pentanoic acid ethyl ester (14 g, 42.9 mmol) in methanol (100 mL) was added pyrrolidine (3.3 g, 47.2 mmol) dropwise. The mixture was then heated at 80° C. for 2 h. After cooled to room temperature, the mixture was concentrated. The residue was purified by flash column to give the title compound (4.2 g).

### Example 50

### Preparation of Intermediate E/Z-6-chloro-3-[5-chloro-2-(1-ethoxycarbonyl-1-propyl-butoxy)-benzylidene]-2-oxo-2,3-dihydro-indole-1-carboxylic acid tert-butyl ester

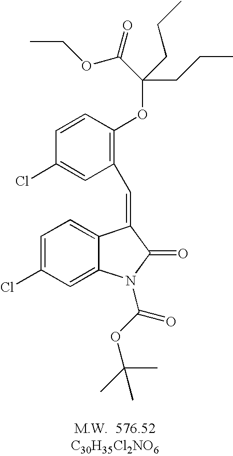

To a solution of E/Z-2-[4-chloro-2-(6-chloro-2-oxo-1,2-dihydro-indol-3-ylidenemethyl)-phenoxy]-2-propyl-pentanoic acid ethyl ester (4.2 g, 8.8 mmol) in dichloromethane (100 mL) at room temperature was added di-tert-butyl-dicarbonate (2.2 g, 9.68 mmol), followed by the addition of 4-dimethylaminopyridine (0.5 g, 4.1 mmol). After the reaction mixture was stirred at room temperature for 1 h, the solution was concentrated. The residue was purified by flash column to give the title compound as a yellow solid (2.6 g).

### Example 51

### Preparation of Intermediate racemic (2′S,3S,4′R)-6-chloro-4′-[5-chloro-2-(1-ethoxycarbonyl-1-propyl-butoxy)-phenyl]-2′-(5-fluoro-2-methyl-phenyl) spiro[3H-indole-3,3′-piperidine]-2,6′(1H)-dione

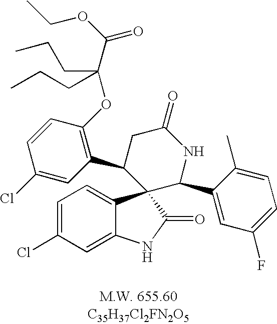

In a manner similar to the method described in Example 5, E/Z-6-chloro-3-[5-chloro-2-(1-ethoxycarbonyl-1-propyl-butoxy)-benzylidene]-2-oxo-2,3-dihydro-indole-1-carboxylic acid tert-butyl ester (1.3 g, 2.3 mmol) was reacted with 1-(5-fluoro-2-methyl-phenyl)-3-trimethylsilyoxy-2-aza-1,3-butadiene (10 mmol) prepared in Example 4 in toluene to give the title compound as a white solid (150 mg).

m/z (M+H)⁺: 655

### Example 52

### Preparation of Intermediate racemic (2′S,3S,4′R)-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione

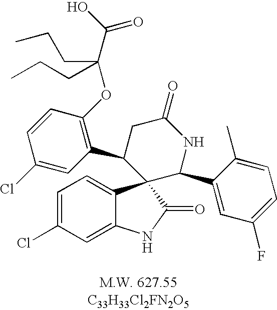

A mixture of racemic (2′S,3S,4′R)-6-chloro-4′-[5-chloro-2-(1-ethoxycarbonyl-1-propyl-butoxy)-phenyl]-2′-(5-fluoro-2-methyl-phenyl) spiro[3H-indole-3,3′-piperidine]-2,6′(1H)-dione (130 mg, 0.198 mmol), LiOH.H₂O (1 g, 24.6 mmol), H₂O (5 mL) and methanol (15 mL) was refluxed for 2 h. After cooled to room temperature, the solution was concentrated and then the aqueous phase was acidified to “pH” 1-2 by addition of concentrated HCl solution and then extracted with EtOAc. The combined organic phases were washed with water and brine, dried over anhydrous Na₂SO₄ and concentrated to give the title compound as a light yellow solid (115 mg).

m/z (M+H)⁺: 627

### Example 53

### Preparation of racemic (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione

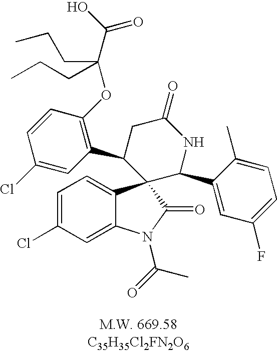

At room temperature, to a mixture of racemic (2′S,3S,4′R)-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione (200 mg, 0.32 mmol) and acetic anhydride (40 mg, 0.38 mmol) in THF (5 mL) was added DMAP (4 mg, 0.033 mmol) slowly. After the mixture was stirred for 0.5 h, the solution was concentrated and the residue was dissolved in EtOAc. The organic layer was washed with 0.5N HCl, dried and concentrated to give the title compound (210 mg).

### Example 54

### Preparation of chiral (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione

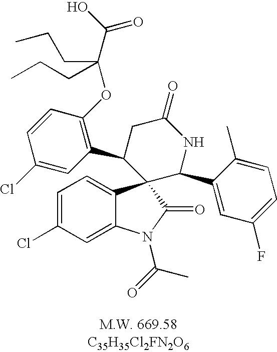

Separation of the two enantiomers from racemic (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione (50 mg), was conducted by chiral SFC to provide chiral (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione as a white solid (11 mg).

m/z (M+H)⁺: 669

### Example 55

### Preparation of chiral (2′R,3R,4′S)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione

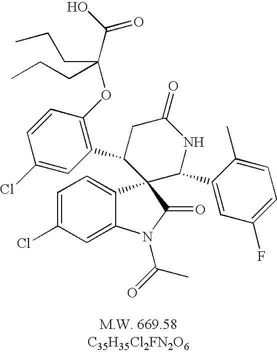

In the SFC chiral separation of the two enantiomers from racemic (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione (50 mg), chiral (2′R,3R,4′S)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione was obtained as a white solid (10 mg).

m/z (M+H)⁺: 669

### Example 56

### Preparation of Intermediate 2-{2-[6-bromo-2-oxo-1,2-dihydro-indol-(3E)-ylidenemethyl]-4-chloro-phenoxy}-2-ethyl-butyric acid methyl ester

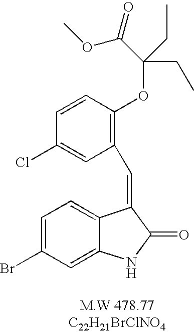

To the mixture of 6-bromooxindole (10.5 g, 49.7 mmol) and 2-(4-chloro-2-formyl-phenoxy)-2-ethyl-butyric acid methyl ester (13 g, 45.8 mmol) in methanol (200 mL) was added pyrrolidine (4.1 mL, 49.7 mmol) dropwise. The mixture was then heated at 70° C. for 2 h. After cooled to room temperature, the mixture was filtered and the precipitate was collected, dried to give the title compound as a yellow solid (16 g).

### Example 57

### Preparation of Intermediate E/Z-6-bromo-3-[1-[5-chloro-2-(1-ethyl-1-methoxycarbonyl-propoxy)-phenyl]-meth-(E)-ylidene]-2-oxo-2,3-dihydro-indole-1-carboxylic acid tert-butyl ester

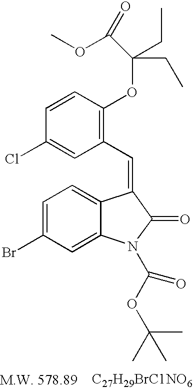

To a solution of E/Z-2-{2-[6-bromo-2-oxo-1,2-dihydro-indol-(3E)-ylidenemethyl]-4-chloro-phenoxy}-2-ethyl-butyric acid methyl ester (16 g, 33.5 mmol) in dichloromethane (200 mL) at room temperature was added di-tert-butyl-dicarbonate (8.6 g, 39 mmol), followed by the addition of 4-dimethylaminopyridine (0.4 g, 3.3 mmol). After the reaction mixture was stirred at room temperature for 1 h, the solution was washed with 1 NHC and brine twice, dried over anhydrous Na₂SO₄ and concentrated to give the title compound as a yellow solid (16 g).

### Example 58

### Preparation of Intermediate racemic (2′S,3S,4′R)-6-bromo-4′-[5-chloro-2-(1-ethyl-1-methoxycarbonyl-propoxy)-phenyl]-2′-(5-fluoro-2-methyl-phenyl) spiro[3H-indole-3,3′-piperidine]-2,6′(1H)-dione

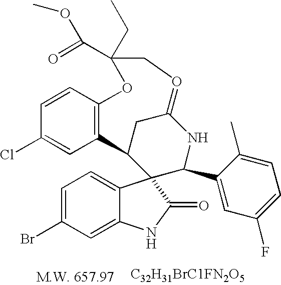

In a manner similar to the method described in Example 5, E/Z-6-bromo-3-[1-[5-chloro-2-(1-ethyl-1-methoxycarbonyl-propoxy)-phenyl]-meth-(E)-ylidene]-2-oxo-2,3-dihydro-indole-1-carboxylic acid tert-butyl ester (6 g, 10 mmol) prepared in Example 57 was reacted with 1-(5-fluoro-2-methyl-phenyl)-3-trimethylsilyoxy-2-aza-1,3-butadiene (40 mmol) prepared in Example 4 in toluene to give the title compound as a white solid (1.2 g).

m/z (M+H)⁺: 657

### Example 59

### Preparation of Intermediate racemic (2′S,3S,4′R)-6-bromo-4′-[5-chloro-2-(1-hydroxycarbonyl-1-ethyl-propoxy)-phenyl]-2′-(5-fluoro-2-methyl-phenyl) spiro[3H-indole-3,3′-piperidine]-2,6′(1H)-dione

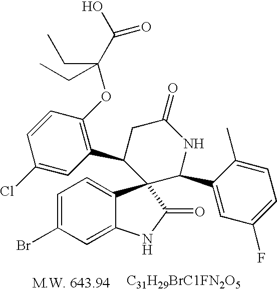

A mixture of racemic (2′S,3S,4′R)-6-bromo-4′-[5-chloro-2-(1-ethyl-1-methoxycarbonyl-propoxy)-phenyl]-2′-(5-fluoro-2-methyl-phenyl) spiro[3H-indole-3,3′-piperidine]-2,6′(1H)-dione (1.2 g, 1.8 mmol), LiOH.H₂O (1.5 g, 36 mmol), H₂O (3 mL) and methanol (10 mL) was refluxed for 2 h. After cooled to room temperature, the solution was concentrated. The water phase was acidified to “pH” 2-3 by addition of concentrated HCl solution and extracted with EtOAc. The combined organic phases were washed with water and brine, dried over anhydrous Na₂SO₄ and concentrated. The residue was washed with methanol to give the title compound (660 mg).

m/z (M+H)⁺: 643

### Example 60

### Preparation of Intermediate racemic (2′S,3S,4′R)-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

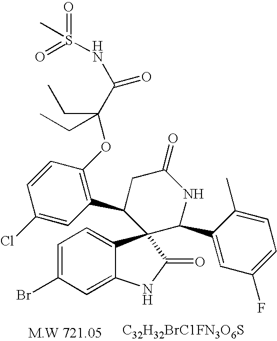

A solution of racemic (2′S,3S,4′R)-6-bromo-4′-[5-chloro-2-(1-hydroxycarbonyl-1-ethyl-propoxy)-phenyl]-2′-(5-fluoro-2-methyl-phenyl) spiro[3H-indole-3,3′-piperidine]-2,6′(1H)-dione (480 mg, 0.75 mmol) and CDI (242 mg, 1.5 mmol) in DMF (5 mL) was heated at 60° C. for 2 h. Then to this solution was added a mixture of methanesulfonamide (712 mg, 7.5 mmol) and NaH (300 mg, 60%, 7.5 mmol) in DMF (5 mL), which had been stirred for 2 h at room temperature. After the resulting mixture was stirred at room temperature for 1 h, it was poured into water and the aqueous solution was acidified by concentrated HCl solution. After the aqueous phase was extracted with EtOAc twice, the combined organic phases were dried over anhydrous Na₂SO₄, concentrated and the residue was purified by flash column to give the title compound as a white solid (300 mg).

m/z (M+H)⁺: 720

### Example 61

### Preparation of racemic (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

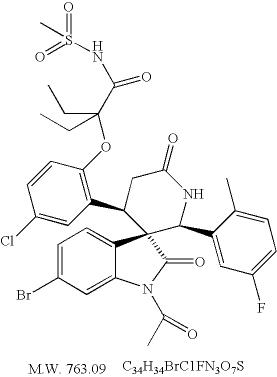

At room temperature, to a mixture of racemic (2′S,3S,4′R)-6-bromo-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione (300 mg, 0.42 mmol) and acetic anhydride (52 mg, 0.50 mmol) in THF (10 mL) was added DMAP (5 mg, 0.04 mmol) slowly. After the mixture was stirred for 2 h, the solution was concentrated. The residue was purified by Prep-HPLC to give the title compound as a white solid (300 mg).

### Example 62

### Preparation of chiral (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

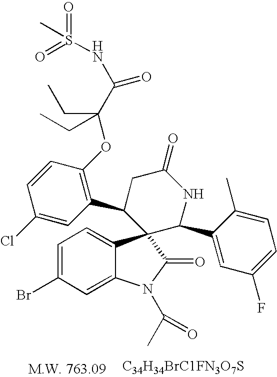

Separation of the two enantiomers from racemic (2′S,3S,4′R)-1-acetyl-6-bromo-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione (50 mg), was conducted by chiral SFC to provide chiral (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione as a white solid (10 mg).

m/z (M+H)⁺: 762

### Example 63

### Preparation of chiral (2′R,3R,4′S)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione

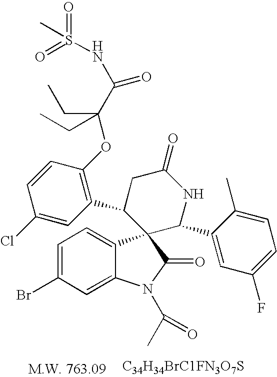

In the SFC chiral separation of the two enantiomers from racemic (2′S,3S,4′R)-1-acetyl-6-bromo-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione (50 mg) in Example 62, chiral (2′R,3R,4′S)-1-acetyl-6-bromo-4′-[5-chloro-2-(1-ethyl-1-methanesulfonylamino carbonyl-propoxy)-phenyl]-2′-(5-chloro-2-methyl-phenyl)-spiro[3H-indole-3,3′-piperidine]-2,6′(1H)-dione was obtained as a white solid (10 mg).

m/z (M+H)⁺: 762

### Example 64

### Preparation of racemic (2′S,3S,4′R)-6-chloro-4′-[5-chloro-2-(1-ethyl-1-methanesulfonylaminocarbonyl-propoxy)-phenyl]-1-(2-ethyl-butyryl)-2′-(5-fluoro-2-methyl-phenyl)-spiro[3H-indole-3,3′-piperidine]-2,6′(1H)-dione

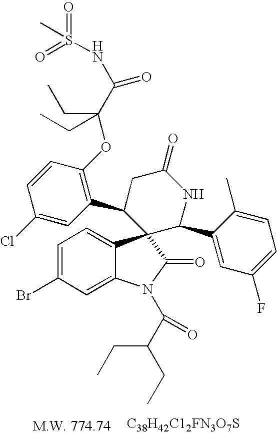

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (680 mg, 1 mmol) prepared in Example 34 and DMAP (183 mg, 1.5 mmol) in THF (10 mL) was added 2-ethyl-butyryl chloride (200 mg, 1.5 mmol) slowly. After the mixture was stirred for 1 h, the solution was concentrated and the residue was purified by flash column to give the title compound (500 mg).

### Example 65

### Preparation of racemic (2′S,3S,4′R)-6-chloro-4′-[5-chloro-2-(1-ethyl-1-methanesulfonylamino carbonyl-propoxy)-phenyl]-2′-(5-fluoro-2-methyl-phenyl)-1-isobutyryl spiro[3H-indole-3,3′-piperidine]-2,6′(1H)-dione

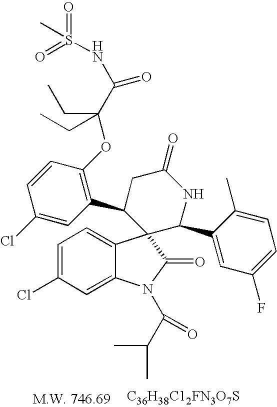

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (680 mg, 1 mmol) prepared in Example 34 and DMAP (183 mg, 1.5 mmol) in THF (10 mL) was added isobutyryl chloride (127 mg, 1.2 mmol) slowly. After the mixture was stirred for 2 h, the solution was concentrated and the residue was purified by flash column to give the title compound (450 mg).

### Example 66

### Preparation of racemic (2′S,3S,4′R)-6-chloro-4′-[5-chloro-2-(1-ethyl-1-methane sulfonylamino carbonyl-propoxy)-phenyl]-2′-(5-fluoro-2-methyl-phenyl)-1-propionyl spiro[3H-indole-3,3′-piperidine]-2,6′(1H)-dione

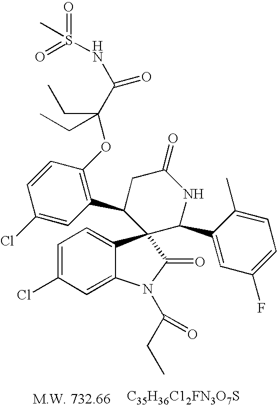

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (68 mg, 0.1 mmol) prepared in Example 34 and propionic anhydride (15.6 mg, 0.12 mmol) in THF (2 mL) was added DMAP (1 mg, 0.008 mmol) slowly. After the mixture was stirred for 4 h, the solution was concentrated. Then methanol was added and the precipitate was collected by filtration to give the title compound (58 mg).

### Example 67

### Preparation of chiral (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione

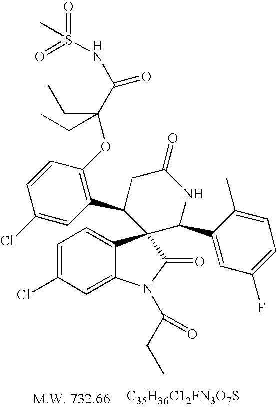

Separation of the two enantiomers from racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione (30 mg), was conducted by chiral SFC to provide chiral (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione as a white solid (10 mg).

m/z (M+H)⁺: 732

### Example 68

### Preparation of chiral (2′R,3R,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione

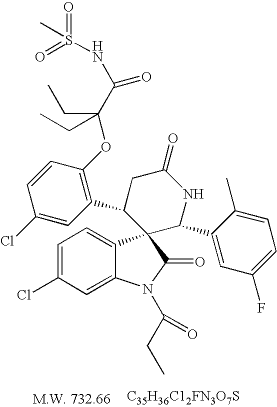

In SFC chiral separation of the two enantiomers from racemic (2′S,3S,4′R)-6-chloro-4′-[5-chloro-2-(1-ethyl-1-methane sulfonylamino carbonyl-propoxy)-phenyl]-2′-(5-fluoro-2-methyl-phenyl)-1-propionyl spiro[3H-indole-3,3′-piperidine]-2,6′(1H)-dione (30 mg) in Example 67, chiral (2′R,3R,4′S)-6-chloro-4′-[5-chloro-2-(1-ethyl-1-methanesulfonylamino carbonyl-propoxy)-phenyl]-2′-(5-fluoro-2-methyl-phenyl)-1-propionyl spiro[3H-indole-3,3′-piperidine]-2,6′(1H)-dione was obtained as a white solid (10 mg).

m/z (M+H)⁺: 732

### Example 69

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isopropoxycarbonyl spiro[3H-indole-3,3′-piperidine]-2,6′(1H)-dione

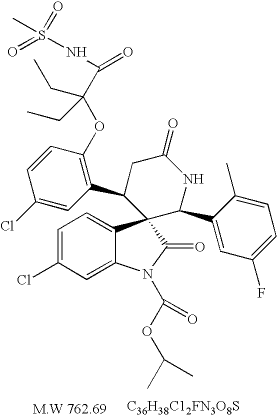

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and isopropyl chloroformate (0.48 mL, 0.48 mmol) in THF (0.5 mL) was added DMAP (99 mg, 0.81 mmol) slowly. After stirred overnight, the mixture was diluted with EtOAc. The solution was washed with 1N HCl solution, dried and concentrated. The residue was purified by flash column to give the title compound (36 mg).

### Example 70

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2-methoxy-ethoxycarbonyl) spiro[3H-indole-3,3′-piperidine]-2,6′(1H)-dione

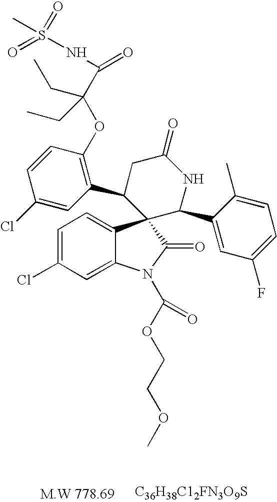

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and 2-methoxyethyl chloroformate (15.3 mg, 0.11 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by flash column to give the title compound as a white solid (29 mg).

### Example 71

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isobutoxycarbonyl spiro[3H-indole-3,3′-piperidine]-2,6′(1H)-dione

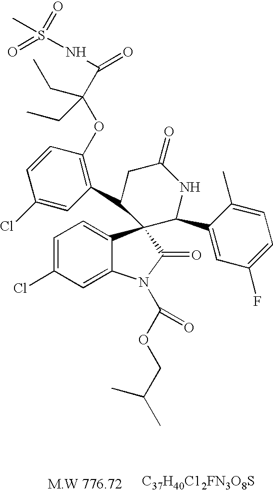

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and isobutyl chloroformate (20 mg, 0.147 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by flash column to give the title compound as a white solid (30 mg).

### Example 72

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-hexyloxycarbonyl spiro[3H-indole-3,3′-piperidine]-2,6′(1H)-dione

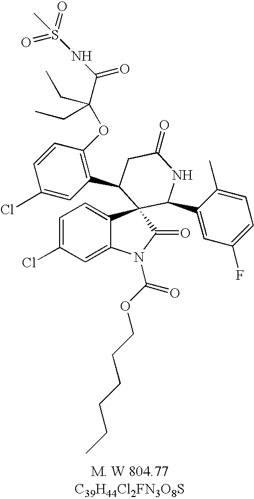

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and hexyl chloroformate (24 mg, 0.146 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by flash column to give the title compound as a white solid (25 mg).

### Example 73

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2,2-dimethyl-propoxycarbonyl) spiro[3H-indole-3,3′-piperidine]-2,6′(1H)-dione

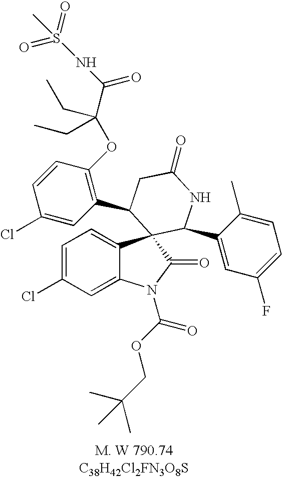

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and neopentyl chloroformate (22 mg, 0.146 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by flash column to give the title compound as a white solid (20 mg).

### Example 74

### Preparation of racemic (2′S,3S,4′R)-6-chloro-4′-[5-chloro-2-(1-ethyl-1-methane sulfonylamino carbonyl-propoxy)-phenyl]-1-(2-fluoro-ethoxycarbonyl)-2′-(5-fluoro-2-methyl-phenyl)-spiro[3H-indole-3,3′-piperidine]-2,6′(1H)-dione

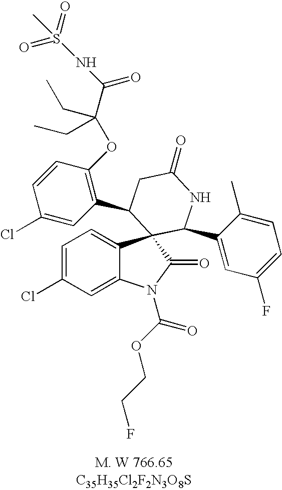

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and neopentyl chloroformate (18 mg, 0.143 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by Prep-HPLC to give the title compound as a white solid (15 mg).

### Example 75

### Preparation of racemic (2′S,3S,4′R)-6-chloro-4′-[5-chloro-2-(1-ethyl-1-methanesulfonylaminocarbonyl-propoxy)-phenyl]-1-ethoxycarbonyl-2′-(5-fluoro-2-methyl-phenyl)-spiro[3H-indole-3,3′-piperidine]-2,6′(1H)-dione

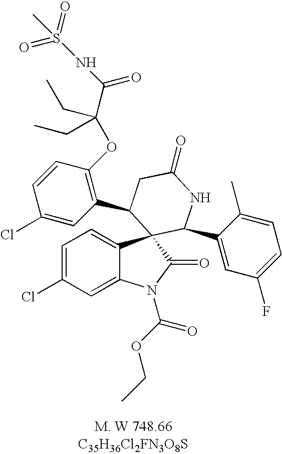

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and ethyl chloroformate (16 mg, 0.148 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by flash column to give the title compound as a white solid (42 mg).

### Example 76

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-methoxycarbonyl spiro[3H-indole-3,3′-piperidine]-2,6′(1H)-dione

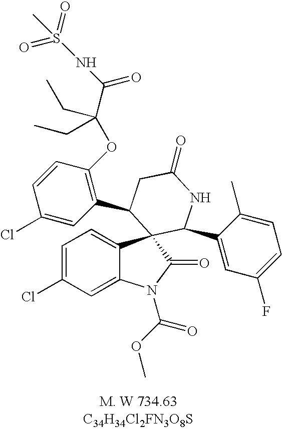

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and methyl chlorocarbonate (14 mg, 0.149 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by Prep-HPLC to give the title compound as a white solid (8 mg).

### Example 77

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propoxycarbonyl spiro[3H-indole-3,3′-piperidine]-2,6′(1H)-dione

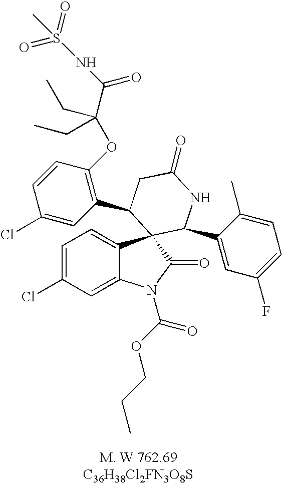

At room temperature, to a mixture of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 and propyl chlorocarbonate (22 mg, 0.18 mmol) in THF (1 mL) was added DMAP (27 mg, 0.22 mmol) slowly. After stirred overnight, the mixture was diluted with EtOAc. The solution was washed with 1N HCl solution, dried and concentrated. The residue was purified by Prep-HPLC to give the title compound as a white solid (9 mg).

### Example 78

### Preparation of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-2,6′-dioxo-2,3-dihydro spiro[indole-3,3′-piperidine]-1-carboxylic acid tert-butyl ester

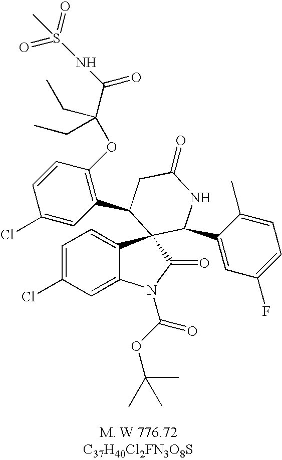

To a solution of racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione (50 mg, 0.074 mmol) prepared in Example 34 in dichloromethane (2 mL) at room temperature was added di-tert-butyl-dicarbonate (24 mg, 0.11 mmol), followed by the addition of 4-dimethylaminopyridine (20 mg, 0.164 mmol). After stirred for 1 h, the mixture was concentrated and the residue was purified by Prep-HPLC to give the title compound (30 mg).

### Example 79

### Preparation of Intermediate E/Z-2-[4-chloro-2-(6-chloro-5-fluoro-2-oxo-1,2-dihydro-indol-3-ylidenemethyl)-phenoxy]-2-ethyl-butyric acid methyl ester

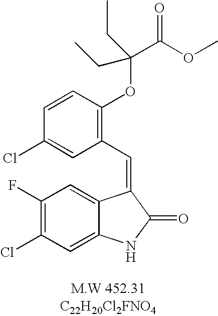

To the mixture of 6-chloro-5-fluoro-1,3-dihydro-indol-2-one (500 mg, 2.7 mmol) and 2-(4-Chloro-2-formyl-phenoxy)-2-ethyl-butyric acid methyl ester (844 mg, 2.97 mmol) (CGENETECH) in methanol (5 mL) was added pyrrolidine (95 mg, 1.35 mmol) dropwise. The mixture was then heated at 70° C. for 1 h. After cooled to room temperature, the mixture was partitioned between EtOAc and diluted HCl. The organic phase was washed with water, brine, dried over anhydrous Na₂SO₄ and concentrated to give the crude product as a red-yellow solid, which was used for the next step reaction without further purification.

### Example 80

### Preparation of Intermediate E/Z-6-chloro-3-[5-chloro-2-(1-ethyl-1-methoxycarbonyl-propoxy)-benzylidene]-5-fluoro-2-oxo-2,3-dihydro-indole-1-carboxylic acid tert-butyl ester

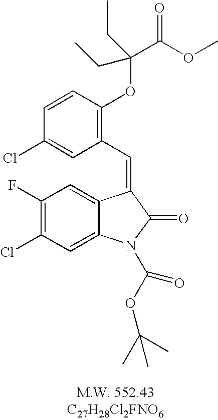

To a solution E/Z-2-[4-chloro-2-(6-chloro-5-fluoro-2-oxo-1,2-dihydro-indol-3-ylidenemethyl)-phenoxy]-2-ethyl-butyric acid methyl ester (1.22 g, 2.7 mmol) in dichloromethane (10 mL) at room temperature was added di-tert-butyl-dicarbonate (0.7 g, 3.24 mmol), followed by the addition of 4-dimethylaminopyridine (0.05 g, 0.41 mmol). After the reaction mixture was stirred at room temperature for 1 h, the solution was concentrated. The residue was purified by flash column to give the title compound as a yellow solid (1.4 g).

### Example 81

### Preparation of Intermediate racemic (2′S,3S,4′R)-6-chloro-4′-[5-chloro-2-(1-methoxycarbonyl-1-ethyl-propoxy)-phenyl]-5-fluoro-2′-(5-fluoro-2-methyl-phenyl) spiro[3H-indole-3,3′-piperidine]-2,6′(1H)-dione

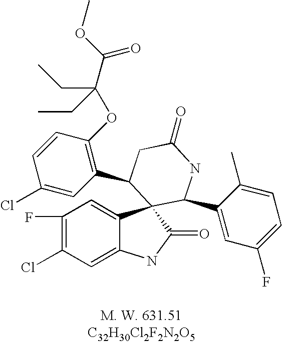

In a manner similar to the method described in Example 5, E/Z-6-chloro-3-[5-chloro-2-(1-ethyl-1-methoxycarbonyl-propoxy)-benzylidene]-5-fluoro-2-oxo-2,3-dihydro-indole-1-carboxylic acid tert-butyl ester (1.4 g, 2.5 mmol) was reacted with 1-(5-fluoro-2-methyl-phenyl)-3-trimethylsilyoxy-2-aza-1,3-butadiene (8 mmol) prepared in Example 4 in toluene (8 mL) to give the title compound (300 mg).

m/z (M+H)⁺: 631

### Example 82

### Preparation of Intermediate racemic (2′S,3S,4′R)-6-chloro-4′-[5-chloro-2-(1-hydroxycarbonyl-1-ethyl-propoxy)-phenyl]-5-fluoro-2′-(5-fluoro-2-methyl-phenyl) spiro[3H-indole-3,3′-piperidine]-2,6′(1H)-dione

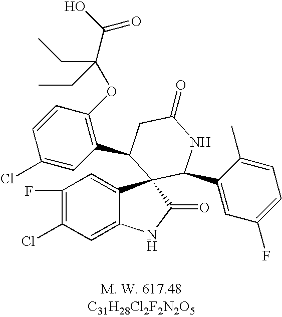

A mixture of racemic (2′S,3S,4′R)-6-chloro-4′-[5-chloro-2-(1-methoxycarbonyl-1-ethyl-propoxy)-phenyl]-5-fluoro-2′-(5-fluoro-2-methyl-phenyl) spiro[3H-indole-3,3′-piperidine]-2,6′(1H)-dione (120 mg, 0.19 mmol), LiOH.H₂O (140 mg, 3.3 mmol), H₂O (1.25 mL) and methanol (3.75 mL) was heated at 80° C. for 1 h. After cooled to room temperature, the mixture was acidified by addition of 0.5 N HCl and partitioned between EtOAc and water. The organic phase was washed with brine, dried over anhydrous Na₂SO₄ and concentrated to give the crude product, which was used for the next step reaction without further purification.

m/z (M+H)⁺: 617

### Example 83

### Preparation of Intermediate racemic (2′S,3S,4′R)-6-chloro-4′-[5-chloro-2-(1-ethyl-1-methanesulfonylaminocarbonyl-propoxy)-phenyl]-5-fluoro-2′-(5-fluoro-2-methyl-phenyl) spiro[3H-indole-3,3′-piperidine]-2,6′(1H)-dione

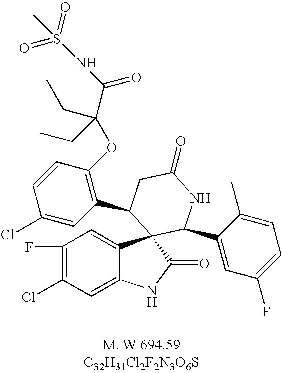

A solution of racemic (2′S,3S,4′R)-6-chloro-4′-[5-chloro-2-(1-hydroxycarbonyl-1-ethyl-propoxy)-phenyl]-5-fluoro-2′-(5-fluoro-2-methyl-phenyl) spiro[3H-indole-3,3′-piperidine]-2,6′(1H)-dione (100 mg, 0.16 mmol) and CDI (123 mg, 0.64 mmol) in DMF (3 mL) was heated at 75° C. for 3 h. Then to this solution was added a mixture of methanesulfonamide (144 mg, 1.5 mmol) and NaH (53 mg, 60%, 1.3 mmol) in DMF (1.5 mL), which had been stirred for 2 h at room temperature. After the resulting mixture was stirred at room temperature for 20 min, it was poured into water and the aqueous solution was acidified by diluted HCl solution. After the aqueous phase was extracted with EtOAc twice, the combined organic phases were dried over anhydrous Na₂SO₄, concentrated and the residue was purified by flash column to give the title compound (50 mg).

m/z (M+H)⁺: 694

### Example 84

### Preparation of racemic (2′S,3S,4′R)-1-acetyl-6-chloro-4′-[5-chloro-2-(1-ethyl-1-methanesulfonylaminocarbonyl-propoxy)-phenyl]-5-fluoro-2′-(5-fluoro-2-methyl-phenyl) spiro[3H-indole-3,3′-piperidine]-2,6′(1H)-dione

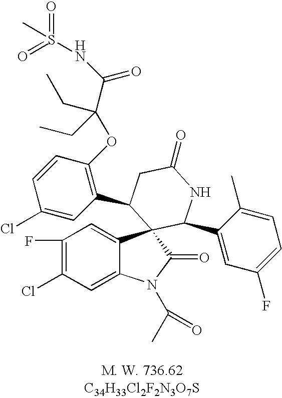

At room temperature, to a mixture of racemic (2′S,3S,4′R)-6-chloro-4′-[5-chloro-2-(1-ethyl-1-methanesulfonylaminocarbonyl-propoxy)-phenyl]-5-fluoro-2′-(5-fluoro-2-methyl-phenyl) spiro[3H-indole-3,3′-piperidine]-2,6′(1H)-dione (30 mg, 0.043 mmol) and acetic anhydride (13 mg, 0.13 mmol) in THF (5 mL) was added DMAP (4.9 mg, 0.04 mmol) slowly. After the mixture was stirred for 0.5 h, the solution was washed by 0.5N HCl solution twice, dried over anhydrous Na₂SO₄ and concentrated to give the title compound (20 mg).

What is claimed:

1. A compound of the formula 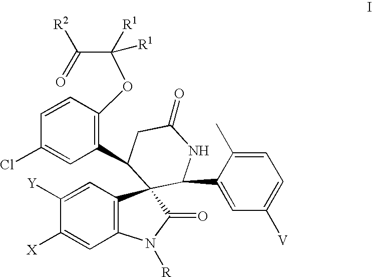
wherein
X is Cl or Br
Y is H or F
V is F or Cl
R¹ is selected from Me, Et or nPr
R² is selected from OH, OMe or NHSO₂Me
R is selected from C(═O)R′, CH₂OH, CH₂OR′ or CH₂C(═O)R′
R′ is selected from lower alkyl, substituted lower alkyl, lower alkoxy, substituted lower alkoxy, cycloalkyl, substituted cycloalkyl, cycloalkoxy or substituted cycloalkoxy.
or a pharmaceutically acceptable salt, ester or enantiomer thereof.

2. The compound of claim 1 wherein
X is Cl,
Y is H,
V is F or Cl,
R¹ is Me or Et,
R² is selected from OH, OMe or NHSO₂Me,
R is C(═O)R′ and
R′ is selected from lower alkyl, substituted lower alkyl, lower alkoxy, substituted lower alkoxy, cycloalkyl, substituted cycloalkyl, cycloalkoxy or substituted cycloalkoxy.

3. A compound of claim 1 selected from the group consisting of
chiral (2′S,3S,4′R)-1-acetyl-6-chloro-4′-[5-chloro-2-(1-hydroxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-6-chloro-4′-[5-chloro-2-(1-methoxycarbonyl-1-methyl-ethoxy)-phenyl]-2,3-dihydro-2′-(5-fluoro-2-methyl-phenyl)-2,6′-dioxo spiro[indole-3,3′-piperidine]-1-carboxylic acid tent-butyl ester;
racemic (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-1-acetyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
racemic (2′S,3S,4′R)-6-chloro-4′-[5-chloro-2-(1-methoxycarbonyl-1-methyl-ethoxy)-phenyl]-2′-(5-fluoro-2-methyl-phenyl)-1-hydroxymethyl spiro[3H-indole-3,3′-piperidine]-2,6′(1H)-dione;
racemic (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-1-acetoxymethyl-6-chloro-4′-[5-chloro-2-(1-methoxycarbonyl-1-methyl-ethoxy)-phenyl]-2′-(5-fluoro-2-methyl-phenyl) spiro[3H-indole-3,3′-piperidine]-2,6′(1H)-dione;
chiral (2′S,3S,4′R)-6-chloro-4′-[5-chloro-2-(1-methoxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione;
racemic (2′S,3S,4′R)-6-chloro-4′-[5-chloro-2-(1-hydroxycarbonyl-1-methyl-ethoxy)-phenyl]-2′-(5-fluoro-2-methylphenyl)-1-(2-trimethylsilanyl-ethoxymethyl) spiro[3H-indole-3,3′-piperidine]-2,6′(1H)-dione and
chiral (2′R,3R,4′S)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione.

4. A compound of claim 1 selected from the group consisting of
chiral (2′S,3S,4′R)-1-acetyl-6-chloro-4′-[5-chloro-2-(2-methanesulfonylamino-1,1-dimethyl-2-oxo-ethoxy)-phenyl]-2′-(5-fluoro-2-methyl-phenyl) spiro[3H-indole-3,3′-piperidine]-2,6′(1H)-dione;
racemic (2′S,3S,4′R)-1-acetyl-6-chloro-4′-[5-chloro-2-(1-ethyl-1-methanesulfonylamino carbonyl-propoxy)-phenyl]-2′-(5-fluoro-2-methyl-phenyl)-spiro[3H-indole-3,3′-piperidine]-2,6′(1H)-dione;
chiral (2′S,3S,4′R)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione;
chiral (2′R,3R,4′S)-1-acetyl-6-chloro-4′-[5-chloro-2-(1-ethyl-1-methanesulfonylaminocarbonyl-propoxy)-phenyl]-2′-(5-fluoro-2-methyl-phenyl)-spiro[3H-indole-3,3′-piperidine]-2,6′(1H)-dione;
racemic (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′S,3S,4′R)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′R,3R,4′S)-1-acetyl-6-chloro-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
racemic (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione;
chiral (2′S,3S,4′R)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione and chiral (2′R,3R,4′S)-1-acetyl-6-chloro-4′-[5-chloro-2-(1-hydroxycarbonyl-1-propyl-butoxy)-phenyl]-2′-(5-fluoro-2-methyl-phenyl) spiro[3H-indole-3,3′-piperidine]-2,6′(1H)-dione.

5. A compound of claim 1 selected from the group consisting of
racemic (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′S,3S,4′R)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
chiral (2′R,3R,4′S)-1-acetyl-6-bromo-4′-[5-chloro-2-(1-ethyl-1-methanesulfonylaminocarbonyl-propoxy)-phenyl]-2′-(5-chloro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-(2-ethyl-butyryl)-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isobutyryl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
chiral (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
chiral (2′R,3R,4′R)-6-chloro-4′-[5-chloro-2-(1-ethyl-1-methanesulfonylaminocarbonyl-propoxy)-phenyl]-2′-(5-fluoro-2-methyl-phenyl)-1-propionyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isopropoxycarbonyl spiro[3H-indole-3,3′-piperidine]-2,6′(1H)-dione and
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2-methoxy-ethoxycarbonyl) spiro[3H-indole-3,3′-piperidine]-2,6′(1H)-dione.

6. A compound of claim 1 selected from the group consisting of
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-isobutoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-hexyloxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-(2,2-dimethyl-propoxycarbonyl) spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-(2-fluoro-ethoxycarbonyl)-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-1-ethoxycarbonyl-2′-(5-fluoro-2-methyl-phenyl)-spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-methoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-1-propoxycarbonyl spiro[3H-indole-3,3′-piperidine]-2,6′(1H)-dione;
racemic (2′S,3S,4′R)-6-chloro-4′-[5-chloro-2-(1-ethyl-1-methanesulfonylaminocarbonyl-propoxy)-phenyl]-2′-(5-fluoro-2-methyl-phenyl)-2,6′-dioxo-2,3-dihydro spiro[indole-3,3′-piperidine]-1-carboxylic acid tert-butyl ester and
racemic (2′S,3S,4′R)-1-acetyl-6-chloro-4′-[5-chloro-2-(1-ethyl-1-methanesulfonylaminocarbonyl-propoxy)-phenyl]-5-fluoro-2′-(5-fluoro-2-methyl-phenyl) spiro[3H-indole-3,3′-piperidine]-2,6′(1H)-dione.

7. A pharmaceutical composition comprising a compound of the formula 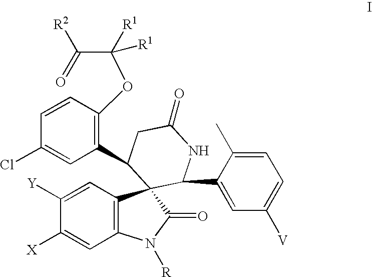
wherein
X is Cl or Br,
Y is H or F,
V is F or Cl,
R¹ is selected from Me, Et or nPr,
R² is selected from OH, Ome or NHSO₂Me,
R is selected from C(═O)R′, or CH₂C(═O)R′ and
R′ is selected from lower alkyl, substituted lower alkyl, lower alkoxy, substituted lower alkoxy, cycloalkyl or substituted cycloalkyl.
or a pharmaceutically acceptable salt, ester or enantiomer thereof together with a pharmaceutically acceptable carrier or excipient.
